(19) United States
(12) Patent Application Publication
(10) Pub. No.: US 2026/0317418 A1
(43) Pub. Date: Oct. 15, 2026

(54) ANTIBODIES HAVING SPECIFICITY TO LTBP2 AND USES THEREOF

(71) Applicants: INSTITUT NATIONAL DE LA SANTÉ ET DE LA RECHERCHE MÉDICALE, Paris (FR); INSTITUT RÉGIONAL DU CANCER DE MONTPELLIER, Montpellier (FR); UNIVERSITÉ DE MONTPELLIER, Montpellier (FR)

(72) Inventors: Andrei TURTOI, Montepllier Cedex 5 (FR); Bruno ROBERT, Montepllier Cedex 5 (FR); Pierre MARTINEAU, Montpellier Cedex 5 (FR); Marie-Alix POUL, Montpellier cedex 5 (FR); Madeline MERLOT, Montpellier Cedex 5 (FR); Evgenia TURTOI, Kleve (DE)

(21) Appl. No.: 19/109,269

(22) PCT Filed: Sep. 7, 2023
(86) PCT No.: PCT/EP2023/074669

§ 371 Date: Mar. 6, 2025

(30) Foreign Application Priority Data
Sep. 8, 2022 (EP) 22306334.8

(51) Int. Cl. C07K 16/28

(52) U.S. Cl. CPC C07K 16/28; C07K 2317/24; C07K 2317/565; C07K 2317/76

(57) ABSTRACT

The present invention relates to the treatment of cancers linked to cancer-associated fibroblasts (CAF). In this study, the inventors have studied the LTBP2+ CAF subset that is responsible for desmoplastic reaction in CRC-LM. They have used gene invalidation experiments to better understand the potential function of these cells, and they have also developed a fully human antibody that enables targeting of LTBP2+ CAF and their depletion in vitro. These new insights identify for the first time a CAF entity within CRC liver metastases whose targeting may be become a valuable asset in the development of new anti-tumor agents. Thus, the present invention relates to antibodies having specificity to LTBP2 and uses thereof.

### FIELD OF THE INVENTION

The present invention relates to antibodies having specificity to LTBP2 and uses thereof.

### BACKGROUND OF THE INVENTION

Metastases account for over 90% of cancer-related deaths worldwide. The liver is a dissemination hub for the deadliest malignancies including colorectal, breast, lung and pancreatic cancers. Over 50% of advanced colorectal cancer (CRC) patients develop liver metastases (CRC-LM) within five years post-primary tumor resection. Only one third of these patients are operable, while the remainder are only eligible for systemic chemotherapy (1,2) with or without targeted therapy. In most cases, this leads to tumor resistance and progression. As a result, the survival of CRC-LM patients rarely exceeds five years.

The tumor microenvironment (TME) of solid tumors offers promising opportunities to treat cancer (3) and the success of immune checkpoint inhibitors (ICIs), e.g., PD-L1 blockade, demonstrates that the disruption of cellular crosstalk between cancer cells and the TME can lead to therapeutic success. However, only a minority of patients benefit from ICIs, especially in CRC (4), indicating that further studies are needed in this area. Cancer-associated fibroblasts (CAFs) are among the most abundant and versatile components of the stroma. They are implicated in all the hallmarks of cancer (5) and growing evidence suggests that CAFs can display both tumor suppressive and promoting functions (6). Indeed, recent single-cell studies in primary breast tumors strengthened the long-held suspicion that CAFs do not adopt a unique phenotype within a single tumor (7,8). Some CAF populations may display immunosuppressive properties while others do not. In addition to their phenotypic heterogeneity, CAFs can derive from multiple sources beyond tissue-resident fibroblasts (9). In the liver, two obvious physiological sources exist: resident (portal) fibroblasts (PFs) and hepatic stellate cells (HSCs) (10). PFs reside in portal spaces, where they produce the connective tissue containing the bile duct, portal vein, and artery-three essential structures for the liver. HSCs are vitamin A storing and lipid droplet containing cells that are found in the Disse space. A considerable body of data shows that HSCs can be activated by liver injury, either through secreted factors or by immune cells directly, including liver resident macrophages (Kupffer cells) (11). However, these findings mainly rely on cell fate tracing studies, which are only possible to perform on mice (12).

A first cell atlas covering human healthy and cirrhotic livers, produced using a single-cell approach (13), showed that at least four populations of mesenchymal cells exist in the liver. Three of these populations showed different degrees of association with response to liver injury: HSCs expressing high levels of RGS5; cells expressing high levels of collagens and PDGFRA but devoid of RGS5 expression; and a population of vascular smooth muscle cells (VSMC) expressing MYH11 strongly. Pericytes expressing RGS5 and VSMCs were identified among lung tumor CAFs in other single-cell reports on lung cancer (14) or multiple primary tumors (15). Two recent reports established first single-cell atlases of CRC-LM composition including CAFs, but their focus was tumor composition change upon chemotherapy (16) or a pan-population description with limited analysis of CAF heterogeneity (17) which is the interest in the present study.

### SUMMARY OF THE INVENTION

In this study, the inventors have studied the LTBP2+ CAF subset that is responsible for desmoplastic reaction in CRC-LM. They have used gene invalidation experiments to better understand the potential function of these cells, and they have also developed a fully human antibody that enables targeting of LTBP2+ CAF and their depletion in vitro. These new insights identify for the first time a CAF entity within CRC liver metastases whose targeting may be become a valuable asset in the development of new anti-tumor agents.

The present invention relates to antibodies having specificity to LTBP2 and uses thereof. In particular, the present invention is defined by the claims.

### DETAILED DESCRIPTION OF THE INVENTION

The present invention relates to fully human antibody having specificity to LTBP-2, particularly LTBP2+ CAF and uses thereof. In particular, the inventors developed 4 fully human antibodies (called also here C6, D2, F5 and F7) against LTBP2 and showed that LTBP2+ CAFs can be depleted targeting LTBP2.

As used herein, the term “LTBP2” for “Latent-transforming growth factor beta-binding protein 2” has its general meaning in the art and denotes a protein of the family of latent transforming growth factor (TGF)-beta binding proteins (LTBP), which are extracellular matrix proteins with multi-domain structure. This protein is the largest member of the LTBP family possessing unique regions and with most similarity to the fibrillins. The amino acid sequence for LTBP2 is: Q14767 (Uniprot) and the nucleic acid sequence for LTBP2 is 4053 (Entrez).

According to the present invention, the VH region of the C6 mab consists of the sequence of SEQ ID NO:1. Accordingly, the H-CDR1 of the C6 mab is defined by the sequence ranging from the amino acid residue at position 31 to the amino acid residue at position 35 in SEQ ID NO:1. Accordingly, the H-CDR2 of C6 mab is defined by the sequence ranging from the amino acid residue at position 50 to the amino acid residue at position 66 in SEQ ID NO:1. Accordingly, the H-CDR3 of the C6 mab is defined by the sequence ranging from the amino acid residue at position 100 to the amino acid residue at position 113 in SEQ ID NO:1.

SEQ ID NO: 1: VH region of the C6 mab
FR1-CDR1-FR2-CDR2-FR3-CDR3-FR4
EVQLVESGGSLVKPGGSLRLSCAASGFTFSNYGMNWVRQAPGKGLEWISS
IYGSSRYISYADFVKGRFTISRDNATNSLYLQMNSLRAEDTAVYYCVRSS
SYNSYYGGGMDVWGRGTLVTVSS

According to the present invention, the VL region of the C6 mab antibody consists of the sequence of SEQ ID NO:2. Accordingly, the L-CDR1 of the C6 mab is defined by the sequence ranging from the amino acid residue at position 23 to the amino acid residue at position 36 in SEQ ID NO:2. Accordingly, the L-CDR2 of the C6 mab is defined by the sequence ranging from the amino acid residue at position 52 to the amino acid residue at position 58 in SEQ ID NO:2. Accordingly, the L-CDR3 of the C6 mab is defined by the sequence ranging from the amino acid residue at position 91 to the amino acid residue at position 100 in SEQ ID NO:2.

SEQ ID NO: 2: VL region of the C6 mab
antibody FR1-CDR1-FR2-CDR2-FR3-CDR3-FR4
QSVLTQPASVSGSPGQSITISCAGTSSDVGGSSYVSWYQQHPGKAPKLMI
YYDSYRPSGVSNRFSGSKSGNTASLTISGLQAEDEADYYCSSNTYSSTRV
FGGGTKLAVL

According to the present invention, the VH region of the D2 mab consists of the sequence of SEQ ID NO:3. Accordingly, the H-CDR1 of the D2 mab is defined by the sequence ranging from the amino acid residue at position 31 to the amino acid residue at position 35 in SEQ ID NO:3. Accordingly, the H-CDR2 of D2 mab is defined by the sequence ranging from the amino acid residue at position 50 to the amino acid residue at position 66 in SEQ ID NO:3. Accordingly, the H-CDR3 of the D2 mab is defined by the sequence ranging from the amino acid residue at position 100 to the amino acid residue at position 108 in SEQ ID NO:3.

SEQ ID NO: 3: VH region of the D2 mab
FR1-CDR1-FR2-CDR2-FR3-CDR3-FR4
EVQLVESGGSLVKPGGSLRLSCAASGFTFSNNYMNWVRQAPGKGLEWISG
IDGSSRGIDYADFVKGRFTISRDNATNSLYLQMNSLRAEDTAVYYCVRSS
SNGGMDVWGRGTLVTVSS

According to the present invention, the VL region of the D2 mab antibody consists of the sequence of SEQ ID NO:4. Accordingly, the L-CDR1 of the D2 mab is defined by the sequence ranging from the amino acid residue at position 23 to the amino acid residue at position 36 in SEQ ID NO:4. Accordingly, the L-CDR2 of the D2 mab is defined by the sequence ranging from the amino acid residue at position 52 to the amino acid residue at position 58 in SEQ ID NO:4. Accordingly, the L-CDR3 of the D2 mab is defined by the sequence ranging from the amino acid residue at position 91 to the amino acid residue at position 100 in SEQ ID NO:4.

SEQ ID NO: 4: VL region of the D2 mab antibody
FR1-CDR1-FR2-CDR2-FR3-CDR3-FR4
QSVLTQPASVSGSPGQSITISCAGTSSDVGGNGYVSWYQQHPGKAPKLMI
YYDSYRPSGVSNRFSGSKSGNTASLTISGLQAEDEADYYCSSYRSGSTRV
FGGGTKLAVL

According to the present invention, the VH region of the F5 mab consists of the sequence of SEQ ID NO:5. Accordingly, the H-CDR1 of the F5 mab is defined by the sequence ranging from the amino acid residue at position 31 to the amino acid residue at position 35 in SEQ ID NO:5. Accordingly, the H-CDR2 of F5 mab is defined by the sequence ranging from the amino acid residue at position 50 to the amino acid residue at position 66 in SEQ ID NO:5. Accordingly, the H-CDR3 of the F5 mab is defined by the sequence ranging from the amino acid residue at position 99 to the amino acid residue at position 107 in SEQ ID NO:5.

SEQ ID NO: 5: VH region of the F5 mab
FR1-CDR1-FR2-CDR2-FR3-CDR3-FR4
EVQLVESGGSLVKPGGSLRLSCAASGFTFSNYDMNWVRQAPGKGLEWISS
ISGSSRDIYYADFVKGRFTISRDNATNSLYLQMNSLRAEDTAVYYCVRSS
YYYGMDVWGRGTLVTVSS

According to the present invention, the VL region of the F5 mab antibody consists of the sequence of SEQ ID NO:6. Accordingly, the L-CDR1 of the F5 mab is defined by the sequence ranging from the amino acid residue at position 23 to the amino acid residue at position 36 in SEQ ID NO:6. Accordingly, the L-CDR2 of the F5 mab is defined by the sequence ranging from the amino acid residue at position 52 to the amino acid residue at position 58 in SEQ ID NO:6. Accordingly, the L-CDR3 of the F5 mab is defined by the sequence ranging from the amino acid residue at position 91 to the amino acid residue at position 100 in SEQ ID NO:6.

SEQ ID NO: 6: VL region of the F5 mab antibody
FR1-CDR1-FR2-CDR2-FR3-CDR3-FR4
QSVLTQPASVSGSPGQSITISCAGTSSDVGGYYGVSWYQQHPGKAPKLMI
YYDSNRPSGVSNRFSGSKSGNTASLTISGLQAEDEADYYCSSYTYNSTRV
FGGGTKLAVL

According to the present invention, the VH region of the F7 mab consists of the sequence of SEQ ID NO:7. Accordingly, the H-CDR1 of the F7 mab is defined by the sequence ranging from the amino acid residue at position 31 to the amino acid residue at position 35 in SEQ ID NO:7. Accordingly, the H-CDR2 of F7 mab is defined by the sequence ranging from the amino acid residue at position 50 to the amino acid residue at position 66 in SEQ ID NO:7. Accordingly, the H-CDR3 of the F7 mab is defined by the sequence ranging from the amino acid residue at position 99 to the amino acid residue at position 109 in SEQ ID NO:7.

SEQ ID NO: 7: VH region of the F7 mab
FR1-CDR1-FR2-CDR2-FR3-CDR3-FR4
EVQLVESGGSLVKPGGSLRLSCAASGFTFSNYSMNWVRQAPGKGLEWISS
ISGSSRSISYADFVKGRFTISRDNATNSLYLQMNSLRAEDTAVYYCVRSS
YYGGNGMDVWGRGTLVTVSS

According to the present invention, the VL region of the F7 mab antibody consists of the sequence of SEQ ID NO:8. Accordingly, the L-CDR1 of the F7 mab is defined by the sequence ranging from the amino acid residue at position 23 to the amino acid residue at position 36 in SEQ ID NO:8. Accordingly, the L-CDR2 of the F7 mab is defined by the sequence ranging from the amino acid residue at position 52 to the amino acid residue at position 58 in SEQ ID NO:8. Accordingly, the L-CDR3 of the F7 mab is defined by the sequence ranging from the amino acid residue at position 91 to the amino acid residue at position 100 in SEQ ID NO:8.

SEQ ID NO: 8: VL region of the F7 mab antibody
FR1-CDR1-FR2-CDR2-FR3-CDR3-FR4
QSVLTQPASVSGSPGQSITISCAGTSSDVGGSYYVSWYQQHPGKAPKLMI
YGDSYRPSGVSNRFSGSKSGNTASLTISGLQAEDEADYYCSSYTYYSTRV
FGGGTKLAVL

As used herein the term “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. In one embodiment, the antibody of the invention is a monoclonal antibody.

In the context of the invention, the amino acid residues of the antibody of the invention are numbered according to the KABAT numbering system. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system. (http://www.bioinf.org.uk/abs/#cdrdef).

As used herein, the term “specificity” refers to the ability of an antibody to detectably bind an epitope presented on an antigen, such as LTBP2, while having relatively little detectable reactivity with non-LTBP2 proteins or structures (such as other proteins presented on cancerous cell, or on other cell types). Specificity can be relatively determined by binding or competitive binding assays, using, e.g., Biacore instruments, as described elsewhere herein. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules (in this case the specific antigen is LTBP2).

The term “affinity”, as used herein, means the strength of the binding of an antibody to an epitope. The affinity of an antibody is given by the dissociation constant Kd, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc, and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of Biacore instruments.

The terms “monoclonal antibody”, “monoclonal Ab”, “monoclonal antibody composition”, “mAb”, or the like, as used herein refer to a preparation of antibody molecules of single molecular composition. A monoclonal antibody composition displays a single binding specificity and affinity for a particular epitope.

The antibodies of the present invention are produced by any technique known in the art, such as, without limitation, any chemical, biological, genetic or enzymatic technique, either alone or in combination. Typically, knowing the amino acid sequence of the desired sequence, one skilled in the art can readily produce said antibodies, by standard techniques for production of polypeptides. For instance, they can be synthesized using well-known solid phase method, preferably using a commercially available peptide synthesis apparatus (such as that made by Applied Biosystems, Foster City, California) and following the manufacturer's instructions. Alternatively, antibodies of the present invention can be synthesized by recombinant DNA techniques well-known in the art. For example, antibodies can be obtained as DNA expression products after incorporation of DNA sequences encoding the antibodies into expression vectors and introduction of such vectors into suitable eukaryotic or prokaryotic hosts that will express the desired antibodies, from which they can be later isolated using well-known techniques.

In one embodiment, the antibody of the invention is an antigen biding fragment selected from the group consisting of a Fab, a F(ab)′2, a single domain antibody, a ScFv, a Sc(Fv)2, a diabody, a triabody, a tetrabody, an unibody, a minibody, a maxibody, a small modular immunopharmaceutical (SMIP), minimal recognition units consisting of the amino acid residues that mimic the hypervariable region of an antibody as an isolated complementary determining region (CDR), and fragments which comprise or consist of the VL or VH chains as well as amino sequence acid having at least 70,71,72,73,74,75,76,77,78,79,80,81,82,83,84,85,86,87,88,89,90,91,92,93,94,95,96,97,98,99 or 100% of identity with SEQ ID NO: 1 to 8.

In some embodiments, the antibody of the present invention is an antibody having a heavy chain comprising i) the H-CDR1 of C6, D2, F5 or F7 mabs, ii) the H-CDR2 of 1 C6, D2, F5 or F7 mabs, and iii) the H-CDR3 of 1 C6, D2, F5 or F7 mabs, and a light chain comprising i) the L-CDR1 of C6, D2, F5 or F7 mabs, ii) the L-CDR2 of C6, D2, F5 or F7 mabs, and iii) the L-CDR3 of C6, D2, F5 or F7 mabs.

In some embodiments, the antibody of the present invention is an antibody having a heavy chain having at least 70 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with SEQ ID NO:1, 3, 5 or 7 and a light chain having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with SEQ ID NO:2, 4, 6 or 8.

Particularly, the antibody has an heavy chain having at least 70% of identity with SEQ ID NO: 1, 3, 5 or 7 and a light chain having at least 70% of identity with SEQ ID NO:2, 4, 6 or 8.

In some embodiments, the antibody of the present invention is an antibody having a heavy chain identical to SEQ ID NO:1, 3, 5 or 7 and a light chain identical to SEQ ID NO:2, 4, 6 or 8.

The term “antigen binding fragment” of an antibody, as used herein, refers to one or more fragments of an intact antibody that retain the ability to specifically binds to a given antigen (e.g., [LTBP2]). Antigen biding functions of an antibody can be performed by fragments of an intact antibody. Examples of biding fragments encompassed within the term antigen biding fragment of an antibody include a Fab fragment, a monovalent fragment consisting of the VL,VH,CL and CH1 domains; a Fab′ fragment, a monovalent fragment consisting of the VL,VH,CL,CH1 domains and hinge region; a F(ab′)2 fragment, a bivalent fragment comprising two Fab′ fragments linked by a disulfide bridge at the hinge region; an Fd fragment consisting of VH domains of a single arm of an antibody; a single domain antibody (sdAb) fragment (Ward et al., 1989 Nature 341:544-546), which consists of a VH domain or a VL domain; and an isolated complementary determining region (CDR). Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (ScFv); see, e.g., Bird et al., 1989 Science 242:423-426; and Huston et al., 1988 proc. Natl. Acad. Sci. 85:5879-5883). “dsFv” is a VH::VL heterodimer stabilised by a disulfide bond. Divalent and multivalent antibody fragments can form either spontaneously by association of monovalent scFvs, or can be generated by coupling monovalent scFvs by a peptide linker, such as divalent sc(Fv)2. Such single chain antibodies include one or more antigen biding portions or fragments of an antibody. These antibody fragments are obtained using conventional techniques known to those skilled in the art, and the fragments are screened for utility in the same manner as are intact antibodies. A unibody is another type of antibody fragment lacking the hinge region of IgG4 antibodies. The deletion of the hinge region results in a molecule that is essentially half the size of traditional IgG4 antibodies and has a univalent binding region rather than the bivalent biding region of IgG4 antibodies. Antigen binding fragments can be incorporated into single domain antibodies, SMIP, maxibodies, minibodies, intrabodies, diabodies, triabodies and tetrabodies (see, e.g., Hollinger and Hudson, 2005, Nature Biotechnology, 23, 9, 1126-1136). The term “diabodies” “tribodies” or “tetrabodies” refers to small antibody fragments with multivalent antigen-binding sites (2, 3 or four), which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Antigen biding fragments can be incorporated into single chain molecules comprising a pair of tandem Fv segments (VH—CH1-VH—CH1) Which, together with complementary light chain polypeptides, form a pair of antigen binding regions (Zapata et al., 1995 Protein Eng. 8 (10); 1057-1062 and U.S. Pat. No. 5,641,870).

The Fab of the present invention can be obtained by treating an antibody which specifically reacts with LTBP2 with a protease, papaine. Also, the Fab can be produced by inserting DNA encoding Fab of the antibody into a vector for prokaryotic expression system, or for eukaryotic expression system, and introducing the vector into a procaryote or eucaryote (as appropriate) to express the Fab.

The F(ab′)2 of the present invention can be obtained treating an antibody which specifically reacts with [antigen] with a protease, pepsin. Also, the F(ab′)2 can be produced by binding Fab′ described below via a thioether bond or a disulfide bond.

The Fab′ of the present invention can be obtained treating F(ab′)2 which specifically reacts with LTBP2 with a reducing agent, dithiothreitol. Also, the Fab′ can be produced by inserting DNA encoding Fab′ fragment of the antibody into an expression vector for prokaryote, or an expression vector for eukaryote, and introducing the vector into a prokaryote or eukaryote (as appropriate) to perform its expression.

The scFv of the present invention can be produced by obtaining cDNA encoding the VH and VL domains as previously described, constructing DNA encoding scFv, inserting the DNA into an expression vector for prokaryote, or an expression vector for eukaryote, and then introducing the expression vector into a prokaryote or eukaryote (as appropriate) to express the scFv. To generate a humanized scFv fragment, a well-known technology called CDR grafting may be used, which involves selecting the complementary determining regions (CDRs) from a donor scFv fragment, and grafting them onto a human scFv fragment framework of known three dimensional structure (see, e.g., WO98/45322; WO 87/02671; U.S. Pat. Nos. 5,859,205; 5,585,089; 4,816,567; EP0173494).

Domain Antibodies (dAbs) are the smallest functional binding units of antibodies-molecular weight approximately 13 kDa—and correspond to the variable regions of either the heavy (VH) or light (VL) chains of antibodies. Further details on domain antibodies and methods of their production are found in U.S. Pat. Nos. 6,291,158; 6,582,915; 6,593,081; 6,172,197; and 6,696,245; US 2004/0110941; EP 1433846, 0368684 and 0616640; WO 2005/035572, 2004/101790, 2004/081026, 2004/058821, 2004/003019 and 2003/002609, each of which is herein incorporated by reference in its entirety.

UniBodies are another antibody fragment technology, based upon the removal of the hinge region of IgG4 antibodies. The deletion of the hinge region results in a molecule that is essentially half the size of a traditional IgG4 antibody and has a univalent binding region rather than a bivalent binding region. Furthermore, because UniBodies are about smaller, they may show better distribution over larger solid tumors with potentially advantageous efficacy. Further details on UniBodies may be obtained by reference to WO 2007/059782, which is incorporated by reference in its entirety.

In a particular embodiment, the antibodies of the invention can be used to treat cancer.

Thus, in a particular embodiment, the invention relates to a method of treating cancer in a subject in need thereof comprising administering to the subject a therapeutically effective amount of the antibodies of the invention.

According to the invention, the cancer may be a liquid or a solid cancer.

In one embodiment, the cancer may be a cancer selected from the group consisting in adrenal cortical cancer, anal cancer, bile duct cancer (e.g. perihilar cancer, distal bile duct cancer, intrahepatic bile duct cancer), bladder cancer, bone cancer (e.g. osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma), brain and central nervous system cancer (e.g. meningioma, astocytoma, oligodendrogliomas, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating lobular carcinoma, lobular carcinoma in situ, gynecomastia), Castleman disease (e.g. giant lymph node hyperplasia, angiofollicular lymph node hyperplasia), cervical cancer, colorectal cancer, endometrial cancer (e.g. endometrial adenocarcinoma, adenocanthoma, papillary serous adenocarcinoma, clear cell), esophagus cancer, gallbladder cancer (mucinous adenocarcinoma, small cell carcinoma), gastrointestinal carcinoid tumors (e.g. choriocarcinoma, chorioadenoma destruens), Hodgkin's disease, Kaposi's sarcoma, kidney cancer (e.g. renal cell cancer), laryngeal and hypopharyngeal cancer, liver cancer (e.g. hemangioma, hepatic adenoma, focal nodular hyperplasia, hepatocellular carcinoma), lung cancer (e.g. small cell lung cancer, non-small cell lung cancer), mesothelioma, plasmacytoma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), salivary gland cancer, skin cancer (e.g. melanoma, nonmelanoma skin cancer), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma), vaginal cancer, vulvar cancer, uterine cancer (e.g. uterine leiomyosarcoma), leukaemia (like acute myeloid leukaemia, acute lymphoid leukaemia, chronic myelomonocytic leukemia (CMML) . . . ), lymphoma and myelodysplastic syndrome (MDS).

### Nucleic Acid Sequence, Vectors and Host Cells

Accordingly, a further object of the invention relates to a nucleic acid molecule encoding any antibodies according to the invention. More particularly the nucleic acid molecule encodes a heavy chain or a light chain of any antibodies of the present invention.

Typically, said nucleic acid is a DNA or RNA molecule, which may be included in any suitable vector, such as a plasmid, cosmid, episome, artificial chromosome, phage or a viral vector. As used herein, the terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. So, a further aspect of the invention relates to a vector comprising a nucleic acid of the invention. Such vectors may comprise regulatory elements, such as a promoter, enhancer, terminator and the like, to cause or direct expression of said antibody upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like. Any expression vector for animal cell can be used, so long as a gene encoding the human antibody C region can be inserted and expressed. Examples of suitable vectors include PAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

A further aspect of the invention relates to a host cell which has been transfected, infected or transformed by a nucleic acid and/or a vector according to the invention.

The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”.

The nucleic acids of the invention may be used to produce an antibody of the present invention in a suitable expression system. The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL·P2·G11·16Ag·20 cell (ATCC CRL1662, hereinafter referred to as “YB2/0 cell”), and the like. The present invention also relates to a method of producing a recombinant host cell expressing an antibody according to the invention, said method comprising the steps of: (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said antibody. Such recombinant host cells can be used for the production of antibodies of the present invention.

Antibodies of the present invention are suitably separated from the culture medium by conventional immunoglobulin purification procedures such as, for example, protein A-Sepharose, hydroxylapatite chromatography, gel electrophoresis, dialysis, or affinity chromatography.

### Functional Variants

The present invention thus provides antibodies comprising functional variants of the VL region, VH region of the antibodies of the invention. A functional variant of a VL or VH used in the context of a monoclonal antibody of the present invention still allows the antibody to retain at least a substantial proportion (at least about 50%, 60%, 70%, 80%, 90%, 95% or more) of the affinity/avidity and/or the specificity/selectivity of the parent antibody (i.e. C6, D2, F5 or F7 mabs) and in some cases such a monoclonal antibody of the present invention may be associated with greater affinity, selectivity and/or specificity than the parent Ab. Such variants can be obtained by a number of affinity maturation protocols including mutating the CDRs (Yang et al., J. Mol. Biol., 254, 392-403, 1995), chain shuffling (Marks et al., Bio/Technology, 10, 779-783, 1992), use of mutator strains of E. coli (Low et al., J. Mol. Biol., 250, 359-368, 1996), DNA shuffling (Patten et al., Curr. Opin. Biotechnol., 8, 724-733, 1997), phage display (Thompson et al., J. Mol. Biol., 256, 77-88, 1996) and sexual PCR (Crameri et al., Nature, 391, 288-291, 1998). Vaughan et al. (supra) discusses these methods of affinity maturation. Such functional variants typically retain significant sequence identity to the parent Ab. The sequence of CDR variants may differ from the sequence of the CDR of the parent antibody sequences through mostly conservative substitutions; for instance at least about 35%, about 50% or more, about 60% or more, about 70% or more, about 75% or more, about 80% or more, about 85% or more, about 90% or more, (e.g., about 65-95%, such as about 92%, 93% or 94%) of the substitutions in the variant are conservative amino acid residue replacements. The sequences of CDR variants may differ from the sequence of the CDRs of the parent antibody sequences through mostly conservative substitutions; for instance at least 10, such as at least 9, 8, 7, 6, 5, 4, 3, 2 or 1 of the substitutions in the variant are conservative amino acid residue replacements. In the context of the present invention, conservative substitutions may be defined by substitutions within the classes of amino acids reflected as follows:
Aliphatic residues I, L, V, and M
Cycloalkenyl-associated residues F, H, W, and Y
Hydrophobic residues A, C, F, G, H, I, L, M, R, T, V, W, and Y
Negatively charged residues D and E
Polar residues C, D, E, H, K, N, Q, R, S, and T
Positively charged residues H, K, and R
Small residues A, C, D, G, N, P, S, T, and V
Very small residues A, G, and S
Residues involved in turn A, C, D, E, G, H, K, N, Q, R, S, P, and formation T
Flexible residues Q, T, K, S, G, P, D, E, and R

More conservative substitutions groupings include: valine-leucine-isoleucine, phenylalanine-tyrosine, lysine-arginine, alanine-valine, and asparagine-glutamine. Conservation in terms of hydropathic/hydrophilic properties and residue weight/size also is substantially retained in a variant CDR as compared to a CDR of [Ab name]. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The retention of similar residues may also or alternatively be measured by a similarity score, as determined by use of a BLAST program (e.g., BLAST 2.2.8 available through the NCBI using standard settings BLOSUM62, Open Gap=11 and Extended Gap=1). Suitable variants typically exhibit at least about 70% of identity to the parent peptide. According to the present invention a first amino acid sequence having at least 70% of identity with a second amino acid sequence means that the first sequence has 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; or 100% of identity with the second amino acid sequence. According to the present invention a first amino acid sequence having at least 90% of identity with a second amino acid sequence means that the first sequence has 90; 91; 92; 93; 94; 95; 96; 97; 98; 99; or 100% of identity with the second amino acid sequence.

In a particular embodiment, the antibodies described above bind to the same antigen and have the same properties of the antibody of the invention i.e. the antibody with the VH and VL of SEQ ID NO: 1 and 2 or 3 and 4 or 5 and 6 or 7 and 8.

### Antibody Which Compete with the Antibody of the Invention

In another aspect, the invention provides an antibody that competes for binding to LTBP2 with the antibodies of the invention.

As used herein, the term “binding” in the context of the binding of an antibody to a predetermined antigen or epitope typically is a binding with an affinity corresponding to a KD of about 10⁻⁷ M or less, such as about 10⁻⁸ M or less, such as about 10⁻⁹ M or less, about 10-10 M or less, or about 10-11 M or even less when determined by for instance surface plasmon resonance (SPR) technology in a BIAcore 3000 instrument using a soluble form of the antigen as the ligand and the antibody as the analyte. BIACORE® (GE Healthcare, Piscaataway, NJ) is one of a variety of surface plasmon resonance assay formats that are routinely used to epitope bin panels of monoclonal antibodies. Typically, an antibody binds to the predetermined antigen with an affinity corresponding to a KD that is at least ten-fold lower, such as at least 100-fold lower, for instance at least 1,000-fold lower, such as at least 10,000-fold lower, for instance at least 100,000-fold lower than its KD for binding to a non-specific antigen (e.g., BSA, casein), which is not identical or closely related to the predetermined antigen. When the KD of the antibody is very low (that is, the antibody has a high affinity), then the KD with which it binds the antigen is typically at least 10,000-fold lower than its KD for a non-specific antigen. An antibody is said to essentially not bind an antigen or epitope if such binding is either not detectable (using, for example, plasmon resonance (SPR) technology in a BIAcore 3000 instrument using a soluble form of the antigen as the ligand and the antibody as the analyte), or is 100 fold, 500 fold, 1000 fold or more than 1000 fold less than the binding detected by that antibody and an antigen or epitope having a different chemical structure or amino acid sequence.

Additional antibodies can be identified based on their ability to cross-compete (e.g., to competitively inhibit the binding of, in a statistically significant manner) with other antibodies of the invention in standard LTBP2 binding assays. The ability of a test antibody to inhibit the binding of antibodies of the present invention to LTBP2 demonstrates that the test antibody can compete with that antibody for binding to LTBP2; such an antibody may, according to non-limiting theory, bind to the same or a related (e.g., a structurally similar or spatially proximal) epitope on LTBP2 as the antibody with which it competes. Thus, another aspect of the invention provides antibodies that bind to the same antigen as, and compete with, the antibodies disclosed herein. As used herein, an antibody “competes” for binding when the competing antibody inhibits LTBP2 binding of an antibody or antigen binding fragment of the invention by more than 50,51,52,53,54,55,56,57,58,59,60,61,62,63,64,65,66,67,68,69,70,71,72,73,74,75,76,77,78,79, 80,81,82,83,84,85,86,87,88,89,90,91,92,93,94,95,96,97,98 or 99% in the presence of an equimolar concentration of competing antibody.

In other embodiments the antibodies or antigen binding fragments of the invention bind to one or more epitopes of LTBP2. In some embodiments, the epitopes to which the present antibodies or antigen binding fragments bind are linear epitopes. In other embodiments, the epitopes to which the present antibodies or antigen binding fragments bind are non-linear, conformational epitopes.

The antibodies of the invention may be assayed for specific binding by any method known in the art. Many different competitive binding assay format(s) can be used for epitope binding. The immunoassays which can be used include, but are not limited to, competitive assay systems using techniques such western blots, radioimmunoassays, ELISA, “sandwich” immunoassays, immunoprecipitation assays, precipitin assays, gel diffusion precipitin assays, immunoradiometric assays, fluorescent immunoassays, protein A immunoassays, and complement-fixation assays. Such assays are routine and well known in the art (see, e.g., Ausubel et al., eds, 1994 Current Protocols in Molecular Biology, Vol. 1, John Wiley & sons, Inc., New York).

### Antibody Engineering

Engineered antibodies of the invention include those in which modifications have been made to framework residues within VH and/or VL, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies are also intended to be encompassed by the invention. Another type of framework modification involves mutating one or more residues within the framework region, or even within one or more CDR regions, to remove T cell-epitopes to thereby reduce the potential immunogenicity of the antibody. This approach is also referred to as “deimmunization” and is described in further detail in U.S. Patent Publication No. 20030153043 by Carr et al.

In some embodiments, the glycosylation of an antibody is modified. Glycosylation can be altered to, for example, increase the affinity of the antibody for the antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

In some embodiments, some mutations are made to the amino acids localized in aggregation “hotspots” within and near the first CDR (CDR1) to decrease the antibodies susceptibility to aggregation (see Joseph M. Perchiacca et al., Proteins 2011; 79:2637-2647).

The antibody of the present invention may be of any isotype. The choice of isotype typically will be guided by the desired effector functions. IgG1 and IgG3 are isotypes that mediate such effectors functions as ADCC or CDC, when IgG2 and IgG4 don't or in a lower manner. Either of the human light chain constant regions, kappa or lambda, may be used. If desired, the class of a monoclonal antibody of the present invention may be switched by known methods. Typical, class switching techniques may be used to convert one IgG subclass to another, for instance from IgG1 to IgG2. Thus, the effector function of the monoclonal antibodies of the present invention may be changed by isotype switching to, e.g., an IgG1, IgG2, IgG3, IgG4, IgD, IgA, IgE, or IgM antibody for various therapeutic uses.

In some embodiments, the antibody of the present invention is a full-length antibody. In some embodiments, the full-length antibody is an IgG1 antibody. In some embodiments, the full-length antibody is an IgG3 antibody.

In some embodiments, the antibody of the present invention is an antibody of a non-IgG2/4 type, e.g. IgG1 or IgG3 which has been mutated such that the ability to mediate effector functions, such as ADCC, has been reduced or even eliminated. Such mutations have e.g. been described in Dall'Acqua W F et al., J Immunol. 177(2): 1129-1138 (2006) and Hezareh M, J Virol. 75(24): 12161-12168 (2001).

In some embodiments, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In some embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In some embodiments, one or more amino acids selected from amino acid residues can be replaced with a different amino acid residue such that the antibody has altered Clq binding and/or reduced or abolished complement dependent cytotoxicity (CDC). This approach is described in further detail in U.S. Pat. No. 6,194,551 by ldusogie et al.

In some embodiments, one or more amino acid residues are altered to thereby alter the ability of the antibody to fix complement. This approach is described further in PCT Publication WO 94/29351 by Bodmer et al.

In some embodiments, the Fc region is modified to increase the ability of the antibody to mediate antibody dependent cellular cytotoxicity (ADCC) and/or to increase the affinity of the antibody for an Fc receptor by modifying one or more amino acids. This approach is described further in PCT Publication WO 00/42072 by Presta. Moreover, the binding sites on human IgG1 for FcγRI, FcγRII, FcγRIII and FcRn have been mapped and variants with improved binding have been described (see Shields, R. L. et al., 2001 J. Biol. Chen. 276:6591-6604, WO2010106180).

The term “antibody-dependent cell-mediated cytotoxicity” or “ADCC” is a term well understood in the art, and refers to a cell-mediated reaction in which non-specific cytotoxic cells that express Fc receptors (FcRs) recognize bound antibody on a target cell and subsequently cause lysis of the target cell. Non-specific cytotoxic cells that mediate ADCC include natural killer (NK) cells, macrophages, monocytes, neutrophils, and eosinophils.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

Additionally or alternatively, an antibody can be made that has an altered type of glycosylation, such as a hypofucosylated or non-fucosylated antibody having reduced amounts of or no fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the present invention to thereby produce an antibody with altered glycosylation. For example, EP1,176,195 by Hang et al. describes a cell line with a functionally disrupted FUT8 gene, which encodes a fucosyl transferase, such that antibodies expressed in such a cell line exhibit hypofucosylation or are devoid of fucosyl residues. Therefore, in some embodiments, the monoclonal antibodies of the present invention may be produced by recombinant expression in a cell line which exhibit hypofucosylation or non-fucosylation pattern, for example, a mammalian cell line with deficient expression of the FUT8 gene encoding fucosyltransferase. PCT Publication WO 03/035835 by Presta describes a variant CHO cell line, Lecl3 cells, with reduced ability to attach fucose to Asn (297)-linked carbohydrates, also resulting in hypofucosylation of antibodies expressed in that host cell (see also Shields, R. L. et al, 2002 J. Biol. Chem. 277:26733-26740). PCT Publication WO 99/54342 by Umana et al. describes cell lines engineered to express glycoprotein-modifying glycosyl transferases (e.g., beta (1,4)-N acetylglucosaminyltransferase III (GnTIII)) such that antibodies expressed in the engineered cell lines exhibit increased bisecting GlcNac structures which results in increased ADCC activity of the antibodies (see also Umana et al, 1999 Nat. Biotech. 17:176-180). Eureka Therapeutics further describes genetically engineered CHO mammalian cells capable of producing antibodies with altered mammalian glycosylation pattern devoid of fucosyl residues (http://www.eurekainc.com/a&boutus/companyoverview.html). Alternatively, the monoclonal antibodies of the present invention can be produced in yeasts or filamentous fungi engineered for mammalian-like glycosylation pattern and capable of producing antibodies lacking fucose as glycosylation pattern (see for example EP1297172B1).

In another embodiment, the antibody is modified to increase its biological half-life. Various approaches are possible. For example, one or more of the following mutations can be introduced: T252L, T254S, T256F, as described in U.S. Pat. No. 6,277,375 by Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Antibodies with increased half lives and improved binding to the neonatal Fc receptor (FcRn), which is responsible for the transfer of maternal IgGs to the foetus (Guyer et al., J. Immunol. 117:587 (1976) and Kim et al., J. immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311,312, 317, 340, 356, 360, 362, 376, 378, 380, 382, 413, 424, or 434, e.g., substitutions of Fc region residue 434 (U.S. Pat. No. 7,371,826).

Another modification of the antibodies herein that is contemplated by the invention is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP0154316 by Nishimura et al. and EP0401384 by Ishikawa et al.

Another modification of the antibodies that is contemplated by the invention is a conjugate or a protein fusion of at least the antigen-binding region of the antibody of the invention to serum protein, such as human serum albumin or a fragment thereof to increase half-life of the resulting molecule. Such approach is for example described in Ballance et al. EP0322094. Another possibility is a fusion of at least the antigen-binding region of the antibody of the invention to proteins capable of binding to serum proteins, such human serum albumin to increase half-life of the resulting molecule. Such approach is for example described in Nygren et al., EP 0 486 525.

Polysialytion is another technology, which uses the natural polymer polysialic acid (PSA) to prolong the active life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the therapeutic protein in the circulation and prevents it from being recognized by the immune system. The PSA polymer is naturally found in the human body. It was adopted by certain bacteria which evolved over millions of years to coat their walls with it. These naturally polysialylated bacteria were then able, by virtue of molecular mimicry, to foil the body's defense system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body.

Another technology includes the use of hydroxyethyl starch (“HES”) derivatives linked to antibodies. HES is a modified natural polymer derived from waxy maize starch and can be metabolized by the body's enzymes. HES solutions are usually administered to substitute deficient blood volume and to improve the rheological properties of the blood. Hesylation of an antibody enables the prolongation of the circulation half-life by increasing the stability of the molecule, as well as by reducing renal clearance, resulting in an increased biological activity. By varying different parameters, such as the molecular weight of HES, a wide range of HES antibody conjugates can be customized.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In certain embodiments of the invention antibodies have been engineered to remove sites of deamidation. Deamidation is known to cause structural and functional changes in a peptide or protein. Deamidation can result in decreased bioactivity, as well as alterations in pharmacokinetics and antigenicity of the protein pharmaceutical. (Anal Chem. 2005 Mar. 1; 77 (5): 1432-9).

In certain embodiments of the invention the antibodies have been engineered to increase pI and improve their drug-like properties. The pI of a protein is a key determinant of the overall biophysical properties of a molecule. Antibodies that have low pIs have been known to be less soluble, less stable, and prone to aggregation. Further, the purification of antibodies with low pI is challenging and can be problematic especially during scale-up for clinical use. Increasing the pI of the anti-LTBP2 antibodies of the invention or fragments thereof improved their solubility, enabling the antibodies to be formulated at higher concentrations (>100 mg/ml). Formulation of the antibodies at high concentrations (e.g. >100 mg/ml) offers the advantage of being able to administer higher doses of the antibodies into eyes of patients via intravitreal injections, which in turn may enable reduced dosing frequency, a significant advantage for treatment of chronic diseases including cardiovascular disorders. Higher pIs may also increase the FcRn-mediated recycling of the IgG version of the antibody thus enabling the drug to persist in the body for a longer duration, requiring fewer injections. Finally, the overall stability of the antibodies is significantly improved due to the higher pi resulting in longer shelf-life and bioactivity in vivo. Preferably, the pI is greater than or equal to 8.2.

Glycosylation modifications can also induce enhanced anti-inflammatory properties of the antibodies by addition of sialylated glycans. The addition of terminal sialic acid to the Fc glycan reduces FcγR binding and converts IgG antibodies to anti-inflammatory mediators through the acquisition of novel binding activities (see Robert M. Anthony et al., J Clin Immunol (2010) 30 (Suppl 1): S9-S14; Kai-Ting C et al., Antibodies 2013, 2, 392-414).

### Antibody Mimetics

In some embodiments, the heavy and light chains, variable regions domains and CDRs that are disclosed can be used to prepare polypeptides that contain antigen binding region that can specifically bind to LTBP2. For example, the CDRs of the C6, D2, F5 or F7 mabs can be incorporated into a molecule (e.g., a polypeptide) covalently or noncovalently to make an immunoadhesion. An immunoadhesion may incorporate the CDRs as part of a larger polypeptide chain, may covalently link the CDRs to another polypeptide chain, or may incorporate the CDRs noncovalently. The CDRs enable the immunoadhesion to bind specifically to a particular antigen of interest (e.g., LTBP2 or epitope thereof).

The terms “polypeptide” and “protein” are used interchangeably herein to refer to a polymer of amino acid residues. The terms apply to amino acid polymers in which one or more amino acid residues is an artificial chemical mimetic of a corresponding naturally occurring amino acid, as well to naturally occurring amino acids polymers and non-naturally occurring amino acid polymers. Unless otherwise indicated, a particular polypeptide sequence also implicitly encompasses conservatively modified variants thereof.

In some embodiments, the antigen biding fragment of the invention is grafted into non-immunoglobulin based antibodies also called antibody mimetics selected from the group consisting of an affibody, an affilin, an affitin, an adnectin, an atrimer, an evasin, a DARPin, an anticalin, an avimer, a fynomer, and a versabody.

The term “antibody mimetic” is intended to refer to molecules capables of mimicking an antibody's ability to bind an antigen, but which are not limited to native antibody structures. Examples of such antibody mimetics include, but are not limited to, Adnectins, Affibodies, DARPins, Anticalins, Avimers, and versabodies, all of which employ binding structures that, while they mimic traditional antibody binding, are generated from and function via distinct mechanisms. Antigen biding fragments of antibodies can be grafted into scaffolds based on polypeptides such as Fibronectin type III (Fn3) (see U.S. Pat. No. 6,703,199, which describes fibronectin polypeptide monobodies). An affibody is well known in the art and refers to affinity proteins based on a 58 amino acid residue protein domain, derived from one of the IgG binding domain of staphylococcal protein A. DARPins (Designed Ankyrin Repeat Proteins) are well known in the art and refer to an antibody mimetic DRP (designed repeat protein) technology developed to exploit the binding abilities of non-antibody proteins. Anticalins are well known in the art and refer to another antibody mimetic technology, wherein the binding specificity is derived from lipocalins. Anticalins may also be formatted as dual targeting protein, called Duocalins. Avimers are well known in the art and refer to another antibody mimetic technology, Avimers are derived from natural A-domain containing protein. Versabodies are well known in the art and refer to another antibody mimetic technology, they are small proteins of 3-5 kDa with >15% cysteines, which form a high disulfide density scaffold, replacing the hydrophobic core the typical proteins have. Such antibody mimetic can be comprised in a scaffold. The term “scaffold” refers to a polypeptide platform for the engineering of new products with tailored functions and characteristics.

In one aspect, the invention pertains to generating non-immunoglobulin-based antibodies also called antibody mimetics using non-immunoglobulins scaffolds onto which CDRs of the invention can be grafted. Known or future non-immunoglobulin frameworks and scaffolds may be employed, as long as they comprise a binding region specific for the target LTBP2 protein.

The fibronectin scaffolds are based on fibronectin type III domain (e.g., the tenth module of the fibronectin type III (10 Fn3 domain)). The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). These fibronectin-based scaffolds are not an immunoglobulin, although the overall fold is closely related to that of the smallest functional antibody fragment, the variable region of the heavy chain, which comprise the entire antigen recognition unit in camel and llama IgG. Because of this structure, the non-immunoglobulin antibody mimics antigen binding properties that are similar in nature and affinity to those of antibodies. These scaffolds can be used in a loop randomisation and shuffling strategy in vitro that is similar to the process of affinity maturation of antibodies in vivo. These fibronectin-based molecules can be used as scaffolds where the loop regions of the molecule can be replaced with CDRs of the invention using standard cloning techniques.

The Ankyrin technology is based on using proteins with Ankyrin derived repeat modules as scaffolds for bearing variable regions which can be used for binding to different targets. The Ankyrin repeat module is a 33 amino acid polypeptide consisting of two anti-parallel α-helices and a β-turn. Binding of the variable regions is mostly optimized by using ribosome display.

Avimers are derived from natural A-domain containing protein such as LRP-1. These domains are used by nature for protein-protein interactions and in human over 250 proteins are structurally based on “A-domains” monomers (2-10) linked via amino acids linkers. Avimers can be created that can bind to the target antigen using the methodology described in, for example, U.S. patent application publication Nos. 20040175756; 20050053973; 20050048512; and 20060008844.

Affibody affinity ligands are small, simple proteins composed of a three-helix bundle based on the scaffold of one of the IgG-binding domains of protein A. protein A is a surface protein form the bacterium Staphylococcus aureus. This scaffold domain consist of 58 amino acids, 13 of which are randomized to generate affibody libraries with a large number of ligand variants (See e.g., U.S. Pat. No. 5,831,012). Affibody molecules mimic antibodies, they have a molecular weight of 6 kDa. In spite of its small size, the binding site of affibody molecules is similar to that of an antibody.

Anticalins are products developed by the company Pieris ProteoLab AG. They are derived from lipocalins, a widespread group of small and robust proteins that are usually involved in the physiological transport or storage of chemically sensitive or insoluble compounds. Several natural lipocalins occur in human tissues or body liquids. The protein architecture is reminiscent of immunoglobulins, with hypervariable loops on top of a rigid framework. However, in contrast with antibodies or their recombinant fragments, lipocalins are composed of a single polypeptide chain with 160 to 180 amino acids residues, being just marginally bigger than a single immunoglobulin domain. The set of four loops, which makes up the binding pocket, shows pronounced structural plasticity and tolerates a variety of side chains. The binding site can can thus be reshaped in a proprietary process in order to recognize prescribed target molecules of different shape with high affinity and specificity. One protein of lipocalin family, the bilin-binding protein (BBP) of Pieris Brassicae has been used to develop anticalins by mutagenizing the set of four loops. One example of a patent application describing anticalins is in PCT Publication No. WO 199916873.

Affilin molecules are small non-immunoglobulin proteins which are designed for specific affinities towards proteins and small molecules. New affilin molecules can be very quickly selected from two libraries, each of which is based on a different human derived scaffold protein. Affilin molecules do not show any structural homology to immunoglobulin proteins. Currently, two affilin scaffolds are employed, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of “ubiquitin-like” proteins are described in WO2004106368.

Versabodies are highly soluble and can be formulated to high concentrations. Versabodies are exceptionally heat stable and offer extended shelf-life. Additional information regarding Versabodies can be found in US 2007/0191272, which is hereby incorporated by reference in its entirety.

The above descriptions of antibody fragment and mimetic technologies is not intended to be comprehensive. A variety of additional technologies including alternative polypeptide-based technologies, such as fusions of complementarity determining regions as outlined in Qui et al., Nature Biotechnology, 25 (8) 921-929 (2007), as well as nucleic acid-based technologies, such as the RNA aptamer technologies described in U.S. Pat. Nos. 5,789,157; 5,864,026; 5,712,375; 5,763,566; 6,013,443; 6,376,474; 6,613,526; 6,114,120; 6,261,774; and 6,387,620; all of which are hereby incorporated by reference, could be used in the context of the instant invention.

### CAR-T Cells

The present invention also provides chimeric antigen receptors (CARs) comprising an antigen binding domain of the antibodies of the present invention. Typically, said chimeric antigen receptor comprises at least one VH and/or VL sequence of the antibodies of the present invention. The chimeric antigen receptor of the present invention also comprises an extracellular hinge domain, a transmembrane domain, and an intracellular T cell signaling domain.

As used herein, the term “chimeric antigen receptor” or “CAR” has its general meaning in the art and refers to an artificially constructed hybrid protein or polypeptide containing the antigen binding domains of an antibody (e.g., scFv) linked to T-cell signaling domains. Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains.

In some embodiments, the invention provides CARs comprising an antigen-binding domain comprising, consisting of, or consisting essentially of, a single chain variable fragment (scFv) of the 14A5.2 mab. In some embodiments, the antigen binding domain comprises a linker peptide. The linker peptide may be positioned between the light chain variable region and the heavy chain variable region.

In some embodiments, the CAR comprises an extracellular hinge domain, a transmembrane domain, and an intracellular T cell signaling domain selected from the group consisting of CD28, 4-1BB, and CD3ξ intracellular domains. CD28 is a T cell marker important in T cell co-stimulation. 4-1BB transmits a potent costimulatory signal to T cells, promoting differentiation and enhancing long-term survival of T lymphocytes. CD32 associates with TCRs to produce a signal and contains immunoreceptor tyrosine-based activation motifs (ITAMs).

In some embodiments, the chimeric antigen receptor of the present invention can be glycosylated, amidated, carboxylated, phosphorylated, esterified, N-acylated, cyclized via, e.g., a disulfide bridge, or converted into an acid addition salt and/or optionally dimerized or polymerized.

The invention also provides a nucleic acid encoding for a chimeric antigen receptor of the present invention. In some embodiments, the nucleic acid is incorporated in a vector as such as described above.

The present invention also provides a host cell comprising a nucleic acid encoding for a chimeric antigen receptor of the present invention. While the host cell can be of any cell type, can originate from any type of tissue, and can be of any developmental stage; the host cell is a T cell, e.g. isolated from peripheral blood lymphocytes (PBL) or peripheral blood mononuclear cells (PBMC). In some embodiments, the T cell can be any T cell, such as a cultured T cell, e.g., a primary T cell, or a T cell from a cultured T cell line, e.g., Jurkat, SupTI, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell can be any type of T cell and can be of any developmental stage, including but not limited to, CD4+/CD8+ double positive T cells, CD4+ helper T cells, e.g., Th2 cells, CD8+ T cells (e.g., cytotoxic T cells), tumor infiltrating cells, memory T cells, naive T cells, and the like. The T cell may be a CD8+ T cell or a CD4+ T cell.

The population of those T cells prepared as described above can be utilized in methods and compositions for adoptive immunotherapy in accordance with known techniques, or variations thereof that will be apparent to those skilled in the art based on the instant disclosure. See, e.g., US Patent Application Publication No. 2003/0170238 to Gruenberg et al; see also U.S. Pat. No. 4,690,915 to Rosenberg. Adoptive immunotherapy of cancer refers to a therapeutic approach in which immune cells with an antitumor reactivity are administered to a tumor-bearing host, with the aim that the cells mediate either directly or indirectly, the regression of an established tumor. Transfusion of lymphocytes, particularly T lymphocytes, falls into this category. Currently, most adoptive immunotherapies are autolymphocyte therapies (ALT) directed to treatments using the patient's own immune cells. These therapies involve processing the patient's own lymphocytes to either enhance the immune cell mediated response or to recognize specific antigens or foreign substances in the body, including the cancer cells. The treatments are accomplished by removing the patient's lymphocytes and exposing these cells in vitro to biologics and drugs to activate the immune function of the cells. Once the autologous cells are activated, these ex vivo activated cells are reinfused into the patient to enhance the immune system to treat cancer. In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin. A treatment-effective amount of cells in the composition is dependent on the relative representation of the T cells with the desired specificity, on the age and weight of the recipient, on the severity of the targeted condition and on the immunogenicity of the targeted Ags. These amount of cells can be as low as approximately 103/kg, preferably 5×103/kg; and as high as 107/kg, preferably 108/kg. The number of cells will depend upon the ultimate use for which the composition is intended, as will the type of cells included therein. For example, if cells that are specific for a particular Ag are desired, then the population will contain greater than 70%, generally greater than 80%, 85% and 90-95% of such cells. For uses provided herein, the cells are generally in a volume of a liter or less, can be 500 ml or less, even 250 ml or 100 ml or less. The clinically relevant number of immune cells can be apportioned into multiple infusions that cumulatively equal or exceed the desired total amount of cells.

In particular the cells of the present invention are particularly suitable for the treatment of cancer. Accordingly, a further object of the present invention relates to a method of treating cancer in a subject in need thereof comprising administering to the subject a therapeutically effective amount of a population of cells of the present invention.

### Multispecific Antibodies

In some embodiments, the invention provides a multispecific antibody comprising a first antigen binding site from an antibody of the present invention molecule described herein above and at least one second antigen binding site.

In some embodiments, the second antigen-binding site is used for recruiting a killing mechanism such as, for example, by binding an antigen on a human effector cell as a BITE (Bispecific T-Cell engager) antibody which is a bispecific scFv2 directed against target antigen and CD3 on T cells described in U.S. Pat. No. 7,235,641, or by binding a cytotoxic agent or a second therapeutic agent. As used herein, the term “effector cell” refers to an immune cell which is involved in the effector phase of an immune response, as opposed to the cognitive and activation phases of an immune response. Exemplary immune cells include a cell of a myeloid or lymphoid origin, for instance lymphocytes (such as B cells and T cells including cytolytic T cells (CTLs)), killer cells, natural killer cells, macrophages, monocytes, mast cells and granulocytes, such as neutrophils, eosinophils and basophils. Some effector cells express specific Fc receptors (FcRs) and carry out specific immune functions. In some embodiments, an effector cell is capable of inducing ADCC, such as a natural killer cell. For example, monocytes, macrophages, which express FcRs, are involved in specific killing of target cells and presenting antigens to other components of the immune system. In some embodiments, an effector cell may phagocytose a target antigen or target cell. The expression of a particular FcR on an effector cell may be regulated by humoral factors such as cytokines. An effector cell can phagocytose a target antigen or phagocytose or lyse a target cell. Suitable cytotoxic agents and second therapeutic agents are exemplified below, and include toxins (such as radiolabeled peptides), chemotherapeutic agents and prodrugs

In some embodiments, the second antigen-binding site binds to an antigen on a human B cell, such as, e.g., CD19, CD20, CD21, CD22, CD23, CD46, CD80, CD138 and HLA-DR.

In some embodiments, the second antigen-binding site binds a tissue-specific antigen, promoting localization of the bispecific antibody to a specific tissue.

In some embodiments, the second antigen-binding site binds to an antigen located on the same type of cell as the LTBP2-expressing cell, typically a tumor-associated antigen (TAA), but has a binding specificity different from that of the first antigen-binding site. Such multi- or bispecific antibodies can enhance the specificity of the tumor cell binding and/or engage multiple effector pathways. Exemplary TAAs include carcinoembryonic antigen (CEA), prostate specific antigen (PSA), RAGE (renal antigen), a-fetoprotein, CAMEL (CTL-recognized antigen on melanoma), CT antigens (such as MAGE-B5, -B6, -C2, -C3, and D; Mage-12; CT10; NY-ESO-1, SSX-2, GAGE, BAGE, MAGE, and SAGE), mucin antigens (e.g., MUC1, mucin-CA125, etc.), ganglioside antigens, tyrosinase, gp75, c-Met, Marti, MelanA, MUM-1, MUM-2, MUM-3, HLA-B7, Ep-CAM or a cancer-associated integrin, such as α5β3 integrin. Alternatively, the second antigen-binding site binds to a different epitope of [antigen]. The second antigen-binding site may alternatively bind an angiogenic factor or other cancer-associated growth factor, such as a vascular endothelial growth factor, a fibroblast growth factor, epidermal growth factor, angiogenin or a receptor of any of these, particularly receptors associated with cancer progression.

In some embodiments, the second antigen-binding site is from a second antibody or ADC of the invention, such as the antibody of the present invention.

Exemplary formats for the multispecific antibody molecules of the invention include, but are not limited to (i) two antibodies cross-linked by chemical heteroconjugation, one with a specificity to [antigen] and another with a specificity to a second antigen; (ii) a single antibody that comprises two different antigen-binding regions; (iii) a single-chain antibody that comprises two different antigen-binding regions, e.g., two scFvs linked in tandem by an extra peptide linker; (iv) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (v) a chemically-linked bispecific (Fab′)2 fragment; (vi) a Tandab, which is a fusion of two single chain diabodies resulting in a tetravalent bispecific antibody that has two binding sites for each of the target antigens; (vii) a flexibody, which is a combination of scFvs with a diabody resulting in a multivalent molecule; (viii) a so called “dock and lock” molecule, based on the “dimerization and docking domain” in Protein Kinase A, which, when applied to Fabs, can yield a trivalent bispecific binding protein consisting of two identical Fab fragments linked to a different Fab fragment; (ix) a so-called Scorpion molecule, comprising, e.g., two scFvs fused to both termini of a human Fab-arm; and (x) a diabody. Another exemplary format for bispecific antibodies is IgG-like molecules with complementary CH3 domains to force heterodimerization. Such molecules can be prepared using known technologies, such as, e.g., those known as Triomab/Quadroma (Trion Pharma/Fresenius Biotech), Knob-into-Hole (Genentech), CrossMAb (Roche) and electrostatically-matched (Amgen), LUZ-Y (Genentech), Strand Exchange Engineered Domain body (SEEDbody) (EMD Serono), Biclonic (Merus) and DuoBody (Genmab A/S) technologies.

In some embodiments, the bispecific antibody is obtained or obtainable via a controlled Fab-arm exchange, typically using DuoBody technology. In vitro methods for producing bispecific antibodies by controlled Fab-arm exchange have been described in WO2008119353 and WO 2011131746 (both by Genmab A/S). In one exemplary method, described in WO 2008119353, a bispecific antibody is formed by “Fab-arm” or “half-molecule” exchange (swapping of a heavy chain and attached light chain) between two monospecific antibodies, both comprising IgG4-like CH3 regions, upon incubation under reducing conditions. The resulting product is a bispecific antibody having two Fab arms which may comprise different sequences. In another exemplary method, described in WO 2011131746, bispecific antibodies of the present invention are prepared by a method comprising the following steps, wherein at least one of the first and second antibodies is the antibody of the present invention: a) providing a first antibody comprising an Fc region of an immunoglobulin, said Fc region comprising a first CH3 region; b) providing a second antibody comprising an Fc region of an immunoglobulin, said Fc region comprising a second CH3 region; wherein the sequences of said first and second CH3 regions are different and are such that the heterodimeric interaction between said first and second CH3 regions is stronger than each of the homodimeric interactions of said first and second CH3 regions; c) incubating said first antibody together with said second antibody under reducing conditions; and d) obtaining said bispecific antibody, wherein the first antibody is the antibody of the present invention and the second antibody has a different binding specificity, or vice versa. The reducing conditions may, for example, be provided by adding a reducing agent, e.g. selected from 2-mercaptoethylamine, dithiothreitol and tris(2-carboxyethyl)phosphine. Step d) may further comprise restoring the conditions to become non-reducing or less reducing, for example by removal of a reducing agent, e.g. by desalting. Preferably, the sequences of the first and second CH3 regions are different, comprising only a few, fairly conservative, asymmetrical mutations, such that the heterodimeric interaction between said first and second CH3 regions is stronger than each of the homodimeric interactions of said first and second CH3 regions. More details on these interactions and how they can be achieved are provided in WO 2011131746, which is hereby incorporated by reference in its entirety. The following are exemplary embodiments of combinations of such assymetrical mutations, optionally wherein one or both Fc-regions are of the IgG1 isotype.

In some embodiments, the first Fc region has an amino acid substitution at a position selected from the group consisting of: 366, 368, 370, 399, 405, 407 and 409, and the second Fc region has an amino acid substitution at a position selected from the group consisting of: 366, 368, 370, 399, 405, 407 and 409, and wherein the first and second Fc regions are not substituted in the same positions.

In some embodiments, the first Fc region has an amino acid substitution at position 405, and said second Fc region has an amino acid substitution at a position selected from the group consisting of: 366, 368, 370, 399, 407 and 409, optionally 409.

In some embodiments, the first Fc region has an amino acid substitution at position 409, and said second Fc region has an amino acid substitution at a position selected from the group consisting of: 366, 368, 370, 399, 405, and 407, optionally 405 or 368.

In some embodiments, both the first and second Fc regions are of the IgG1 isotype, with the first Fc region having a Leu at position 405, and the second Fc region having an Arg at position 409.

### Immunoconjugates

An antibody of the invention can be conjugated with a detectable label to form an anti-LTBP2 immunoconjugate. Suitable detectable labels include, for example, a radioisotope, a fluorescent label, a chemiluminescent label, an enzyme label, a bioluminescent label or colloidal gold. Methods of making and detecting such detectably-labeled immunoconjugates are well-known to those of ordinary skill in the art, and are described in more detail below. The detectable label can be a radioisotope that is detected by autoradiography. Isotopes that are particularly useful for the purpose of the invention are 3H, 1251, 1311, 35S and 14C.

Anti-LTBP2 immunoconjugates can also be labeled with a fluorescent compound. The presence of a fluorescently-labeled antibody is determined by exposing the immunoconjugate to light of the proper wavelength and detecting the resultant fluorescence. Fluorescent labeling compounds include fluorescein isothiocyanate, rhodamine, phycoerytherin, phycocyanin, allophycocyanin, o-phthaldehyde and fluorescamine.

Alternatively, anti-LTBP2 immunoconjugates can be detectably labeled by coupling an antibody to a chemiluminescent compound. The presence of the chemiluminescent-tagged immunoconjugate is determined by detecting the presence of luminescence that arises during the course of a chemical reaction. Examples of chemiluminescent labeling compounds include luminol, isoluminol, an aromatic acridinium ester, an imidazole, an acridinium salt and an oxalate ester.

Similarly, a bioluminescent compound can be used to label anti-LTBP2 immunoconjugates of the invention. Bioluminescence is a type of chemiluminescence found in biological systems in which a catalytic protein increases the efficiency of the chemiluminescent reaction. The presence of a bioluminescent protein is determined by detecting the presence of luminescence. Bioluminescent compounds that are useful for labeling include luciferin, luciferase and aequorin.

Alternatively, anti-LTBP2 immunoconjugates can be detectably labeled by linking an anti-[antigen] antibody to an enzyme. When the anti-LTBP2-enzyme conjugate is incubated in the presence of the appropriate substrate, the enzyme moiety reacts with the substrate to produce a chemical moiety which can be detected, for example, by spectrophotometric, fluorometric or visual means. Examples of enzymes that can be used to detectably label polyspecific immunoconjugates include β-galactosidase, glucose oxidase, peroxidase and alkaline phosphatase.

Those of skill in the art will know of other suitable labels which can be employed in accordance with the invention. The binding of marker moieties to anti-LTBP2 monoclonal antibodies can be accomplished using standard techniques known to the art. Typical methodology in this regard is described by Kennedy et al., Clin. Chim. Acta 70:1, 1976; Schurs et al., Clin. Chim. Acta 81:1, 1977; Shih et al., Int'l J. Cancer 46:1101, 1990; Stein et al., Cancer Res. 50:1330, 1990; and Coligan, supra.

Moreover, the convenience and versatility of immunochemical detection can be enhanced by using anti-LTBP2 monoclonal antibodies that have been conjugated with avidin, streptavidin, and biotin. (See, e.g., Wilchek et al. (eds.), “Avidin-Biotin Technology,” Methods In Enzymology (Vol. 184) (Academic Press 1990); Bayer et al., “Immunochemical Applications of Avidin-Biotin Technology,” in Methods In Molecular Biology (Vol. 10) 149-162 (Manson, ed., The Humana Press, Inc. 1992).)

Methods for performing immunoassays are well-established. (See, e.g., Cook and Self, “Monoclonal Antibodies in Diagnostic Immunoassays,” in Monoclonal Antibodies: Production, Engineering, and Clinical Application 180-208 (Ritter and Ladyman, eds., Cambridge University Press 1995); Perry, “The Role of Monoclonal Antibodies in the Advancement of Immunoassay Technology,” in Monoclonal Antibodies: Principles and Applications 107-120 (Birch and Lennox, eds., Wiley-Liss, Inc. 1995); Diamandis, Immunoassay (Academic Press, Inc. 1996).)

In some embodiments, the antibody of the present invention is conjugated to a therapeutic moiety, i.e. a drug. The therapeutic moiety can be, e.g., a cytotoxin, a chemotherapeutic agent, a cytokine, an immunosuppressant, an immune stimulator, a lytic peptide, or a radioisotope. Such conjugates are referred to herein as an “antibody-drug conjugates” or “ADCs”.

In some embodiments, the antibody is conjugated to a cytotoxic moiety. The cytotoxic moiety may, for example, be selected from the group consisting of taxol; cytochalasin B; gramicidin D; ethidium bromide; emetine; mitomycin; etoposide; tenoposide; vincristine; vinblastine; colchicin; doxorubicin; daunorubicin; dihydroxy anthracin dione; a tubulin-inhibitor such as maytansine or an analog or derivative thereof; an antimitotic agent such as monomethyl auristatin E or F or an analog or derivative thereof; dolastatin 10 or 15 or an analogue thereof; irinotecan or an analogue thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof, an antimetabolite such as methotrexate, 6 mercaptopurine, 6 thioguanine, cytarabine, fludarabin, 5 fluorouracil, decarbazine, hydroxyurea, asparaginase, gemcitabine, or cladribine; an alkylating agent such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C; a platinum derivative such as cisplatin or carboplatin; duocarmycin A, duocarmycin SA, rachelmycin (CC-1065), or an analog or derivative thereof; an antibiotic such as dactinomycin, bleomycin, daunorubicin, doxorubicin, idarubicin, mithramycin, mitomycin, mitoxantrone, plicamycin, anthramycin (AMC)); pyrrolo[2,1-c][1,4]-benzodiazepines (PDB); diphtheria toxin and related molecules such as diphtheria A chain and active fragments thereof and hybrid molecules, ricin toxin such as ricin A or a deglycosylated ricin A chain toxin, cholera toxin, a Shiga-like toxin such as SLT I, SLT II, SLT IIV, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins such as PAPI, PAPII, and PAP-S, Momordica charantia inhibitor, curcin, crotin, sapaonaria officinalis inhibitor, gelonin, mitogellin, restrictocin, phenomycin, and enomycin toxins; ribonuclease (RNase); DNase I, Staphylococcal enterotoxin A; pokeweed antiviral protein; diphtherin toxin; and Pseudomonas endotoxin.

In some embodiments, the antibody is conjugated to a nucleic acid or nucleic acid-associated molecule. In one such embodiment, the conjugated nucleic acid is a cytotoxic ribonuclease (RNase) or deoxy-ribonuclease (e.g., DNase I), an antisense nucleic acid, an inhibitory RNA molecule (e.g., a siRNA molecule) or an immunostimulatory nucleic acid (e.g., an immunostimulatory CpG motif-containing DNA molecule). In some embodiments, the antibody is conjugated to an aptamer or a ribozyme.

In some embodiments, the antibody is conjugated, e.g., as a fusion protein, to a lytic peptide such as CLIP, Magainin 2, mellitin, Cecropin and P18.

In some embodiments, the antibody is conjugated to a cytokine, such as, e.g., IL-2, IL-4, IL-6, IL-7, IL-10, IL-12, IL-13, IL-15, IL-18, IL-23, IL-24, IL-27, IL-28a, IL-28b, IL-29, KGF, IFNa, IFN3, IFNy, GM-CSF, CD40L, Flt3 ligand, stem cell factor, ancestim, and TNFa.

In some embodiments, the antibody is conjugated to a radioisotope or to a radioisotope-containing chelate. For example, the antibody can be conjugated to a chelator linker, e.g. DOTA, DTPA or tiuxetan, which allows for the antibody to be complexed with a radioisotope. The antibody may also or alternatively comprise or be conjugated to one or more radiolabeled amino acids or other radiolabeled molecules Non-limiting examples of radioisotopes include 3H, 14C, 15N, 35S, 90Y, 99Tc, 1251, 1311, 186Re, 213Bi, 225Ac and 227Th. For therapeutic purposes, a radioisotope emitting beta- or alpha-particle radiation can be used, e.g., 1311, 90Y, 211At, 212Bi, 67Cu, 186Re, 188Re, and 212Pb.

In certain embodiments, an antibody-drug conjugate comprises an anti-tubulin agent. Examples of anti-tubulin agents include, for example, taxanes (e.g., Taxol® (paclitaxel), Taxotere® (docetaxel)), T67 (Tularik), vinca alkyloids (e.g., vincristine, vinblastine, vindesine, and vinorelbine) and dolastatins (e.g., auristatin E, AFP, MMAF, MMAE, AEB, AEVB). Other antitubulin agents include, for example, baccatin derivatives, taxane analogs (e.g., epothilone A and B), nocodazole, colchicine and colcimid, estramustine, cryptophysins, cemadotin, maytansinoids, combretastatins, discodermolide, and eleutherobin. In some embodiments, the cytotoxic agent is a maytansinoid, another group of anti-tubulin agents. For example, in specific embodiments, the maytansinoid is maytansine or DM-1 (ImmunoGen, Inc.; see also Chari et al., Cancer Res. 52:127-131, 1992).

In other embodiments, the cytotoxic agent is an antimetabolite. The antimetabolite can be, for example, a purine antagonist (e.g., azothioprine or mycophenolate mofetil), a dihydrofolate reductase inhibitor (e.g., methotrexate), acyclovir, gangcyclovir, zidovudine, vidarabine, ribavarin, azidothymidine, cytidine arabinoside, amantadine, dideoxyuridine, iododeoxyuridine, poscarnet, or trifluridine.

In other embodiments, an anti-LTBP2 antibody is conjugated to a pro-drug converting enzyme. The pro-drug converting enzyme can be recombinantly fused to the antibody or chemically conjugated thereto using known methods. Exemplary pro-drug converting enzymes are carboxypeptidase G2, β-glucuronidase, penicillin-V-amidase, penicillin-G-amidase, β-lactamase, β-glucosidase, nitroreductase and carboxypeptidase A.

Other molecule using as therapeutic moiety can be PyrroloBenzoDiazepine dimers (PBD).

In a particular embodiment, the antibody is a chimeric antibody having a heavy chain identical to SEQ ID NO:1 and a light chain identical to SEQ ID NO:2 and conjugated to the MMAE.

In another particular embodiment, the antibody is a chimeric antibody having a heavy chain identical to SEQ ID NO:1 and a light chain identical to SEQ ID NO:2 and conjugated to PyrroloBenzoDiazepine dimers (PBD).

Typically, the antibody-drug conjugate compounds comprise a linker unit between the drug unit and the antibody unit. In some embodiments, the linker is cleavable under intracellular conditions, such that cleavage of the linker releases the drug unit from the antibody in the intracellular environment. In yet other embodiments, the linker unit is not cleavable and the drug is released, for example, by antibody degradation.

In some embodiments, the linker is cleavable by a cleaving agent that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123).

Most typical are peptidyl linkers that are cleavable by enzymes that are present in 191P4D12-expressing cells. Examples of such linkers are described, e.g., in U.S. Pat. No. 6,214,345, incorporated herein by reference in its entirety and for all purposes. In a specific embodiment, the peptidyl linker cleavable by an intracellular protease is a Val-Cit linker or a Phe-Lys linker (see, e.g., U.S. Pat. No. 6,214,345, which describes the synthesis of doxorubicin with the Val-Cit linker). One advantage of using intracellular proteolytic release of the therapeutic agent is that the agent is typically attenuated when conjugated and the serum stabilities of the conjugates are typically high.

In other embodiments, the cleavable linker is pH-sensitive, i.e., sensitive to hydrolysis at certain pH values.

Typically, the pH-sensitive linker hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929).

In yet other embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio) propionate), SPDB (N-succinimidyl-3-(2-pyridyldithio) butyrate) and SMPT (N-succinimidyl-oxycarbonyl-alpha-methyl-alpha-(2-pyridyl-dithio) toluene), SPDB and SMPT. (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

In yet other specific embodiments, the linker is a malonate linker (Johnson et al., 1995, Anticancer Res. 15:1387-93), a maleimidobenzoyl linker (Lau et al., 1995, Bioorg-Med-Chem. 3 (10): 1299-1304), or a 3′-N-amide analog (Lau et al., 1995, Bioorg-Med-Chem. 3 (10): 1305-12).

In yet other embodiments, the linker unit is not cleavable and the drug is released by antibody degradation.

Typically, the linker is not substantially sensitive to the extracellular environment. As used herein, “not substantially sensitive to the extracellular environment,” in the context of a linker, means that no more than about 20%, typically no more than about 15%, more typically no more than about 10%, and even more typically no more than about 5%, no more than about 3%, or no more than about 1% of the linkers, in a sample of antibody-drug conjugate compound, are cleaved when the antibody-drug conjugate compound is present in an extracellular environment (e.g., in plasma). Whether a linker is not substantially sensitive to the extracellular environment can be determined, for example, by incubating with plasma the antibody-drug conjugate compound for a predetermined time period (e.g., 2, 4, 8, 16, or 24 hours) and then quantitating the amount of free drug present in the plasma.

Techniques for conjugating molecules to antibodies, are well-known in the art (See, e.g., Arnon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624.) Typically, the nucleic acid molecule is covalently attached to lysines or cysteines on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M., Hutchins, B. M., Kim, C. H., Kazane, S. A., Halder, R., Forsyth, J. S., Santidrian, A. F., Stafin, K., et al. (2012). Synthesis of site-specific antibody-drug conjugates using unnatural amino acids. Proc. Natl. Acad. Sci. USA 109, 16101-16106; Junutula, J. R., Flagella, K. M., Graham, R. A., Parsons, K. L., Ha, E., Raab, H., Bhakta, S., Nguyen, T., Dugger, D. L., Li, G., et al. (2010). Engineered thio-trastuzumab-DM1 conjugate with an improved therapeutic index to target humanepidermal growth factor receptor 2-positive breast cancer. Clin. Cancer Res.16, 4769-4778). Junutula et al. (2008) developed cysteine-based site-specific conjugation called “THIOMABs” (TDCs) that are claimed to display an improved therapeutic index as compared to conventional conjugation methods. Conjugation to unnatural amino acids that have been incorporated into the antibody is also being explored for ADCs; however, the generality of this approach is yet to be established (Axup et al., 2012). In particular the one skilled in the art can also envisage Fc-containing polypeptide engineered with an acyl donor glutamine-containing tag (e.g., Gin-containing peptide tags or Q-tags) or an endogenous glutamine that are made reactive by polypeptide engineering (e.g., via amino acid deletion, insertion, substitution, or mutation on the polypeptide). Then a transglutaminase, can covalently crosslink with an amine donor agent (e.g., a small molecule comprising or attached to a reactive amine) to form a stable and homogenous population of an engineered Fc-containing polypeptide conjugate with the amine donor agent being site-specifically conjugated to the Fc-containing polypeptide through the acyl donor glutamine-containing tag or the accessible/exposed/reactive endogenous glutamine (WO 2012059882).

### Therapeutic Uses

In a further aspect, the invention relates to a LTBP2 inhibitor for use in the treatment of a cancer linked to cancer-associated fibroblasts (CAF) in a subject in need thereof.

As used herein and according to the invention, the term “cancer-associated fibroblasts” denotes a cell type within the tumor microenvironment that promotes tumorigenic features by initiating the remodeling of the extracellular matrix or by secreting cytokines.

A used herein and according to the invention, the term “cancer linked to cancer-associated fibroblasts” or “CAF-rich tumors” denotes any cancer where the action of CAFs are preponderant notably in their pro-angiogenic and extracellular matrix (ECM) organization effects. Cancer linked to cancer-associated fibroblasts are for example colorectal cancer, liver cancer, pancreatic cancer, breast cancer and their associated liver metastases.

In a particular embodiment, the cancer linked to cancer-associated fibroblasts (CAF) is a cancer harboring a subpopulation of CAF denoted as LTBP2+ CAFs.

As used herein, the term “LTBP2 inhibitor” denotes a molecule or compound which can inhibit the interactions of the LTBP2 with the microenvironment, or a molecule or compound which destabilizes LTBP2. The term “LTBP2 inhibitor” also denotes an inhibitor of the expression of the gene coding for the protein. In the context of the invention, using a LTBP2 inhibitor and particularly an antibody anti-LTBP2, LTBP2+ CAFs can be depleted. More particularly, the inventors showed that LTBP2 can also be expressed in cancer cells (see the results part). Anti-LTBP2 can thus be used to inhibit the progression/invasion of cancer and notably aggressive cancers like HCC.

LTBP2 inhibitors are well known in the state of the art including siRNA and shRNA (Pang X. F et al., 2019, Acta Physiol, DOI:10.111/alpha. 13377 & Wan F et al., 2016, Oncol Res, DOI:10.3727/096504016X14755368915591).

According to the invention, the LTBP2 inhibitors can be the antibodies of the invention (C6, D2, F5 or F7 mabs, see above).

In one embodiment, the inhibitor according to the invention may be a low molecular weight compound, e.g. a small organic molecule (natural or not). The term “small organic molecule” refers to a molecule (natural or not) of a size comparable to those organic molecules generally used in pharmaceuticals. The term excludes biological macromolecules (e.g., proteins, nucleic acids, etc.). Preferred small organic molecules range in size up to about 10000 Da, more preferably up to 5000 Da, more preferably up to 2000 Da and most preferably up to about 1000 Da.

In one embodiment, the compound according to the invention is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996). Then, for this invention, neutralizing aptamers of LTBP2 are selected.

In one embodiment, the compound according to the invention is a polypeptide. In a particular embodiment the polypeptide is an antagonist of LTBP2 and is capable to prevent the function of LTBP2. Particularly, the polypeptide can be a mutated LTBP2 protein or a similar protein without the function of LTBP2. In one embodiment, the polypeptide of the invention may be linked to a cell-penetrating peptide” to allow the penetration of the polypeptide in the cell. The term “cell-penetrating peptides” are well known in the art and refers to cell permeable sequence or membranous penetrating sequence such as penetratin, TAT mitochondrial penetrating sequence and compounds (Bechara and Sagan, 2013; Jones and Sayers, 2012; Khafagy el and Morishita, 2012; Malhi and Murthy, 2012). The polypeptides of the invention may be produced by any suitable means, as will be apparent to those of skill in the art. In order to produce sufficient amounts of polypeptide or functional equivalents thereof for use in accordance with the present invention, expression may conveniently be achieved by culturing under appropriate conditions recombinant host cells containing the polypeptide of the invention. Preferably, the polypeptide is produced by recombinant means, by expression from an encoding nucleic acid molecule. Systems for cloning and expression of a polypeptide in a variety of different host cells are well known. When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E. coli. In specific embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain. Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications. Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 60 kDa). In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

In another embodiment, the LTBP2 inhibitor according to the invention is an inhibitor of LTBP2 gene expression.

Small inhibitory RNAs (siRNAs) can also function as inhibitors of LTBP2 expression for use in the present invention. LTBP2 gene expression can be reduced by contacting a subject or cell with a small double stranded RNA (dsRNA), or a vector or construct causing the production of a small double stranded RNA, such that LTBP2 gene expression is specifically inhibited (i.e. RNA interference or RNAi). Methods for selecting an appropriate dsRNA or dsRNA-encoding vector are well known in the art for genes whose sequence is known (e.g. see for example Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of LTBP2 gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of LTBP2 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays. Both antisense oligonucleotides and ribozymes useful as inhibitors of LTBP2 gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing LTBP2. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles are provided in Kriegler, 1990 and in Murry, 1991. Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, eye, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a particular embodiment, the antisense oligonucleotide, siRNA, shRNA or ribozyme nucleic acid sequence is under the control of a heterologous regulatory region, e.g., a heterologous promoter. The promoter can also be, e.g., a viral promoter, such as CMV promoter or any synthetic promoters.

Particularly, the invention relates to antibodies, fragments or immunoconjugates of the invention for use in the treatment of a cancer linked to cancer-associated fibroblasts in a subject in need thereof.

The antibodies of the invention may be used alone or in combination with any suitable agent.

In each of the embodiments of the treatment methods described herein, the anti-LTBP2 antibody or anti-LTBP2 antibody-drug conjugate is delivered in a manner consistent with conventional methodologies associated with management of the disease or disorder for which treatment is sought. In accordance with the disclosure herein, an effective amount of the antibody or antibody-drug conjugate is administered to a patient in need of such treatment for a time and under conditions sufficient to prevent or treat the disease or disorder.

As used herein, the terms “treatment” and “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “therapeutically effective amount” or “effective amount” refers to an amount effective, at dosages and for periods of time necessary, to achieve a desired therapeutic result. A therapeutically effective amount of the antibody of the present invention may vary according to factors such as the disease state, age, sex, and weight of the individual, and the ability of the antibody of the present invention to elicit a desired response in the individual. A therapeutically effective amount is also one in which any toxic or detrimental effects of the antibody or antibody portion are outweighed by the therapeutically beneficial effects. The efficient dosages and dosage regimens for the antibody of the present invention depend on the disease or condition to be treated and may be determined by the persons skilled in the art. A physician having ordinary skill in the art may readily determine and prescribe the effective amount of the pharmaceutical composition required. For example, the physician could start doses of the antibody of the present invention employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. Typically, the ability of a compound to inhibit cancer may, for example, be evaluated in an animal model system predictive of efficacy in human tumors. Alternatively, this property of a composition may be evaluated by examining the ability of the compound to induce cytotoxicity by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of an antibody of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of an antibody of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. In some embodiments, the efficacy may be monitored by visualization of the disease area, or by other diagnostic methods described further herein, e.g. by performing one or more PET-CT scans, for example using a labeled antibody of the present invention, fragment or mini-antibody derived from the antibody of the present invention. If desired, an effective daily dose of a pharmaceutical composition may be administered as two, three, four, five, six or more sub-doses administered separately at appropriate intervals throughout the day, optionally, in unit dosage forms. In some embodiments, the monoclonal antibodies of the present invention are administered by slow continuous infusion over a long period, such as more than 24 hours, in order to minimize any unwanted side effects. An effective dose of an antibody of the present invention may also be administered using a weekly, biweekly or triweekly dosing period. The dosing period may be restricted to, e.g., 8 weeks, 12 weeks or until clinical progression has been established. As non-limiting examples, treatment according to the present invention may be provided as a daily dosage of an antibody of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

Accordingly, one object of the present invention relates to a method of treating a cancer linked to cancer-associated fibroblasts in a subject in need thereof comprising administering to the subject a therapeutically effective amount of an inhibitor of LTBP2 of the present invention.

Particularly, the inhibitor of LTBP2 is an antibody anti-LTBP2 (C6, D2, F5 or F7 mabs, see above).

In order to test the functionality of a putative LTBP2 inhibitor a test is necessary. For that purpose, to identify LTBP2 inhibitors one can use cell attachment/adhesion assay. CAF treated with LTBP2 inhibitors (LTBP2 blocking/suppressing agents) (e.g. siRNA, antibodies) will detach from the culture support and die.

Other methods for selecting appropriate inhibitor can be used. For example, LTBP2 inhibitors are identified by measurement of the LTBP2 concentration in a liquid (blood, serum, plasma) before and after depletion/inhibition of this liquid with the tested compound/molecule. Then, LTBP2 is detected using standard protocols such as ELISA or Luminex and the propriety of inhibition of the tested compound/molecule is determined.

In another aspect, the present invention relates to the antibody of the present invention, as defined in any aspect or embodiment herein, for use as a medicament.

In certain embodiments, an anti-LTBP2 antibody or a LTBP2 inhibitor is used in combination with a second agent for treatment of a disease or disorder. When used for treating a cancer linked to cancer-associated fibroblasts (or CAF-rich tumors), an anti-LTBP2 antibody of the invention may be used in combination with conventional cancer therapies such as, e.g., surgery, radiotherapy, chemotherapy, or combinations thereof. This is particularly justified in CAF-rich tumors, where drug penetration is hampered by the extracellular matrix (ECM) production of the CAF. There, LTBP2 targeting may decrease the ECM and hence allow a better penetration of the drug in the tumor and hence better therapeutic effect.

The present invention also provides for therapeutic applications where an antibody of the present invention is used in combination with at least one further therapeutic agent, e.g. for treating cancers and metastatic cancers. Such administration may be simultaneous, separate or sequential. For simultaneous administration the agents may be administered as one composition or as separate compositions, as appropriate. The further therapeutic agent is typically relevant for the disorder to be treated. Exemplary therapeutic agents include other anti-cancer antibodies, cytotoxic agents, chemotherapeutic agents, anti-angiogenic agents, anti-cancer immunogens, cell cycle control/apoptosis regulating agents, hormonal regulating agents, and other agents described below.

In some embodiments, the antibody of the present invention is used in combination with a chemotherapeutic agent. The term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In some embodiments, the antibody of the present invention is used in combination with a targeted cancer therapy. Targeted cancer therapies are drugs or other substances that block the growth and spread of cancer by interfering with specific molecules (“molecular targets”) that are involved in the growth, progression, and spread of cancer. Targeted cancer therapies are sometimes called “molecularly targeted drugs,” “molecularly targeted therapies,” “precision medicines,” or similar names. In some embodiments, the targeted therapy consists of administering the subject with a tyrosine kinase inhibitor. The term “tyrosine kinase inhibitor” refers to any of a variety of therapeutic agents or drugs that act as selective or non-selective inhibitors of receptor and/or non-receptor tyrosine kinases. Tyrosine kinase inhibitors and related compounds are well known in the art and described in U.S. Patent Publication 2007/0254295, which is incorporated by reference herein in its entirety. It will be appreciated by one of skill in the art that a compound related to a tyrosine kinase inhibitor will recapitulate the effect of the tyrosine kinase inhibitor, e.g., the related compound will act on a different member of the tyrosine kinase signaling pathway to produce the same effect as would a tyrosine kinase inhibitor of that tyrosine kinase. Examples of tyrosine kinase inhibitors and related compounds suitable for use in methods of embodiments of the present invention include, but are not limited to, dasatinib (BMS-354825), PP2, BEZ235, saracatinib, gefitinib (Iressa), sunitinib (Sutent; SU11248), erlotinib (Tarceva; OSI-1774), lapatinib (GW572016; GW2016), canertinib (CI 1033), semaxinib (SU5416), vatalanib (PTK787/ZK222584), sorafenib (BAY 43-9006), imatinib (Gleevec; STI571), leflunomide (SU101), vandetanib (Zactima; ZD6474), MK-2206 (8-[4-aminocyclobutyl)phenyl]-9-phenyl-1,2,4-triazolo[3,4-f][1,6]naphthyridin-3 (2H)-one hydrochloride) derivatives thereof, analogs thereof, and combinations thereof. Additional tyrosine kinase inhibitors and related compounds suitable for use in the present invention are described in, for example, U.S. Patent Publication 2007/0254295, U.S. Pat. Nos. 5,618,829, 5,639,757, 5,728,868, 5,804,396, 6,100,254, 6,127,374, 6,245,759, 6,306,874, 6,313,138, 6,316,444, 6,329,380, 6,344,459, 6,420,382, 6,479,512, 6,498,165, 6,544,988, 6,562,818, 6,586,423, 6,586,424, 6,740,665, 6,794,393, 6,875,767, 6,927,293, and 6,958,340, all of which are incorporated by reference herein in their entirety. In some embodiments, the tyrosine kinase inhibitor is a small molecule kinase inhibitor that has been orally administered and that has been the subject of at least one Phase I clinical trial, more preferably at least one Phase II clinical, even more preferably at least one Phase III clinical trial, and most preferably approved by the FDA for at least one hematological or oncological indication. Examples of such inhibitors include, but are not limited to, Gefitinib, Erlotinib, Lapatinib, Canertinib, BMS-599626 (AC-480), Neratinib, KRN-633, CEP-11981, Imatinib, Nilotinib, Dasatinib, AZM-475271, CP-724714, TAK-165, Sunitinib, Vatalanib, CP-547632, Vandetanib, Bosutinib, Lestaurtinib, Tandutinib, Midostaurin, Enzastaurin, AEE-788, Pazopanib, Axitinib, Motasenib, OSI-930, Cediranib, KRN-951, Dovitinib, Seliciclib, SNS-032, PD-0332991, MKC-I (Ro-317453; R-440), Sorafenib, ABT-869, Brivanib (BMS-582664), SU-14813, Telatinib, SU-6668, (TSU-68), L-21649, MLN-8054, AEW-541, and PD-0325901.

In some embodiments, the antibody of the present invention is used in combination with an immunotherapeutic agent. The term “immunotherapeutic agent,” as used herein, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non-cytokine adjuvants. Alternatively the immunotherapeutic treatment may consist of administering the subject with an amount of immune cells (T cells, NK, cells, dendritic cells, B cells . . . ). Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents. Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants. A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors. Interferons (IFNs) contemplated by the present invention include the common types of IFNs, IFN-alpha (IFN-α), IFN-beta (IFN-β) and IFN-gamma (IFN-γ). IFNs can act directly on cancer cells, for example, by slowing their growth, promoting their development into cells with more normal behavior and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation). Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin). Combination compositions and combination administration methods of the present invention may also involve “whole cell” and “adoptive” immunotherapy methods. For instance, such methods may comprise infusion or re-infusion of immune system cells (for instance tumor-infiltrating lymphocytes (TILs), such as CC2+ and/or CD8+ T cells (for instance T cells expanded with tumor-specific antigens and/or genetic enhancements), antibody-expressing B cells or other antibody-producing or- presenting cells, dendritic cells (e.g., dendritic cells cultured with a DC-expanding agent such as GM-CSF and/or Flt3-L, and/or tumor-associated antigen-loaded dendritic cells), anti-tumor NK cells, so-called hybrid cells, or combinations thereof. Cell lysates may also be useful in such methods and compositions. Cellular “vaccines” in clinical trials that may be useful in such aspects include Canvaxin™, APC-8015 (Dendreon), HSPPC-96 (Antigenics), and Melacine® cell lysates. Antigens shed from cancer cells, and mixtures thereof (see for instance Bystryn et al., Clinical Cancer Research Vol. 7, 1882-1887 July 2001), optionally admixed with adjuvants such as alum, may also be components in such methods and combination compositions.

Particularly, the antibody of the invention may be used in combination with another antibody like the antibody Ha22-2 (Seattle Genetics) described in the patent application WO2012047724.

In some embodiments, the antibody of the present invention is used in combination with radiotherapy. Radiotherapy may comprise radiation or associated administration of radiopharmaceuticals to a patient. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the antibody of the present invention is used in combination with an antibody that is specific for a costimulatory molecule. Examples of antibodies that are specific for a costimulatory molecule include but are not limited to anti-CTLA4 antibodies (e.g. Ipilimumab), anti-PD1 antibodies, anti-PDL1 antibodies, anti-TIMP3 antibodies, anti-LAG3 antibodies, anti-B7H3 antibodies, anti-B7H4 antibodies or anti-B7H6 antibodies.

In some embodiments, the second agent is an agent that induces, via ADCC, the death of a cell expressing an antigen to which the second agent binds. In some embodiments, the agent is an antibody (e.g. of IgG1 or IgG3 isotype) whose mode of action involves induction of ADCC toward a cell to which the antibody binds. NK cells have an important role in inducing ADCC and increased reactivity of NK cells can be directed to target cells through use of such a second agent. In some embodiments, the second agent is an antibody specific for a cell surface antigens, e.g., membrane antigens. In some embodiments, the second antibody is specific for a tumor antigen as described above (e.g., molecules specifically expressed by tumor cells), such as CD20, CD52, ErbB2 (or HER2/Neu), CD33, CD22, CD25, MUC-1, CEA, KDR, αVβ3, etc., particularly lymphoma antigens (e.g., CD20). Accordingly, the present invention also provides methods to enhance the anti-tumor effect of monoclonal antibodies directed against tumor antigen(s). In the methods of the invention, ADCC function is specifically augmented, which in turn enhances target cell killing, by sequential administration of an antibody directed against one or more tumor antigens, and an antibody of the present invention.

Accordingly, a further object relates to a method of enhancing NK cell antibody-dependent cellular cytotoxicity (ADCC) of an antibody in a subject in need thereof comprising administering to the subject the antibody, and administering to the subject an antibody of the present invention.

A further object of the present invention relates to a method of treating a cancer linked to cancer-associated fibroblasts in a subject in need thereof comprising administering to the subject a first antibody selective for a cancer cell antigen and administering to the subject an antibody of the present invention.

A number of antibodies are currently in clinical use for the treatment of cancer, and others are in varying stages of clinical development. Antibodies of interest for the methods of the invention act through ADCC, and are typically selective for tumor cells, although one of skill in the art will recognize that some clinically useful antibodies do act on non-tumor cells, e.g. CD20. There are a number of antigens and corresponding monoclonal antibodies for the treatment of B cell malignancies. One popular target antigen is CD20, which is found on B cell malignancies. Rituximab is a chimeric unconjugated monoclonal antibody directed at the CD20 antigen. CD20 has an important functional role in B cell activation, proliferation, and differentiation. The CD52 antigen is targeted by the monoclonal antibody alemtuzumab, which is indicated for treatment of chronic lymphocytic leukemia. CD22 is targeted by a number of antibodies, and has recently demonstrated efficacy combined with toxin in chemotherapy-resistant hairy cell leukemia. Monoclonal antibodies targeting CD20, also include tositumomab and ibritumomab. Monoclonal antibodies useful in the methods of the invention, which have been used in solid tumors, include without limitation edrecolomab and trastuzumab (herceptin). Edrecolomab targets the 17-1 A antigen seen in colon and rectal cancer, and has been approved for use in Europe for these indications. Its antitumor effects are mediated through ADCC, CDC, and the induction of an anti-idiotypic network. Trastuzumab targets the HER-2/neu antigen. This antigen is seen on 25% to 35% of breast cancers. Trastuzumab is thought to work in a variety of ways: downregulation of HER-2 receptor expression, inhibition of proliferation of human tumor cells that overexpress HER-2 protein, enhancing immune recruitment and ADCC against tumor cells that overexpress HER-2 protein, and downregulation of angiogenesis factors. Alemtuzumab (Campath) is used in the treatment of chronic lymphocytic leukemia; colon cancer and lung cancer; Gemtuzumab (Mylotarg) finds use in the treatment of acute myelogenous leukemia; Ibritumomab (Zevalin) finds use in the treatment of non-Hodgkin's lymphoma; Panitumumab (Vectibix) finds use in the treatment of colon cancer. Cetuximab (Erbitux) is also of interest for use in the methods of the invention. The antibody binds to the EGF receptor (EGFR), and has been used in the treatment of solid tumors including colon cancer and squamous cell carcinoma of the head and neck (SCCHN).

### Kit of Part

A fifth aspect of the present invention relates to i) a LTBP2 inhibitor, and ii) at least one anti-cancer agent, as a combined preparation for simultaneous, separate or sequential use in the treatment of a cancer linked to cancer-associated fibroblasts.

In some embodiment, the present invention relates to i) an antibody of the invention directed against LTBP2 (C6, D2, F5 or F7 mabs, see above), and ii) at least one anti-cancer agent, as a combined preparation for simultaneous, separate or sequential use in the treatment of a cancer with preponderant stroma.

In some embodiment, the at least one anti-cancer agent is gemcitabine and/or FOLFIRINOX.

As used herein, the term “simultaneous use” denotes the use of a LTBP2 inhibitor and at least one anti-cancer agent occurring at the same time.

As used herein, the term “separate use” denotes the use of a LTBP2 inhibitor and at least one anti-cancer agent not occurring at the same time.

As used herein, the term “sequential use” denotes the use of a LTBP2 inhibitor and at least one anti-cancer agent occurring by following an order.

### Pharmaceutical Compositions

Typically, the antibodies or the inhibitor of the present invention is administered to the subject in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a patient, the composition will be formulated for administration to the patient. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m2 and 500 mg/m2. However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of an anti-myosin 18A antibody of the invention.

In certain embodiments, the use of liposomes and/or nanoparticles is contemplated for the introduction of antibodies into host cells. The formation and use of liposomes and/or nanoparticles are known to those of skill in the art.

Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles that meet these requirements are contemplated for use in the present invention, and such particles may be are easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The physical characteristics of liposomes depend on pH, ionic strength and the presence of divalent cations.

The invention will be further illustrated by the following figure and example. However, these examples and figures should not be interpreted in any way as limiting the scope of the present invention.

### FIGURES

FIG. 1. LTBP2 is essential to the viability of CAF and less/not cancer cells. A) MTT assay showing the proliferation of CCD18Co cells exposed to HT29 conditioned medium (CM) and treated with different antibodies against LTBP2 (F5, C6, F7, D2), or an irrelevant antibody (Rituximab, anti-CD20) for 96h. B) same as in A) on HT29 cancer cells. Means are normalized to the non-treated condition (NT). Error bars represent the standard deviation. P-values were obtained performing a t-test.

FIG. 2. Expression of LTBP2 in HCC

Immunohistochemical analysis of LTBP2 expression in human HCC, its stromal and cancer cell component. Shown are two magnifications (A) and (B) at 100× and 400× respectively. Dark color denotes positivity of LTBP2 expression. Image (B) shows primarily the selective LTBP2 positivity in certain cancer cells.

FIG. 3. LTBP2 Enhances HCC Invasion In Vitro

Study of the invasion capacity of HCC cells engineered to express LTBP2, in particular HLE cell (A) and ALEX cell (B).

FIG. 4. Anti-LTBP2-D2 Comparison to Rituximab and Cetuximab

Study of the LTBP2 inhibitor, the Rituximab (negative control) and the Cetuximab (positive control) on the invasion of HLE cell expressing LTBP2 (A) and on the invasion of ALEX cell expressing LTBP2 (B).

### EXAMPLE

### Material & Methods

### Patient Material

The Translational study committee of the Regional Cancer Hospital ICM, Montpellier has approved the present study. In accordance with French law, patients have provided written consent approving the use of their material for research purposes. Liver metastases originating from 25 colorectal cancer patients (CRC-LM) were used in the present study. All patients were treated with neo-adjuvant chemotherapy prior to surgery and sample collection. Six different tumors were analyzed from five patients using single-cell RNAseq. Four patients had mono-focal liver metastases, while one had bi-focal metastases. The remaining cohort of CRC-LM patients was implicated in the validation study.

### Tumor Collection and Dissociation

Fresh primary tumor and liver metastases were dissected into multiple pieces (10-15 mm2), with care being taken to avoid surrounding non-tumoral tissue as well as visibly necrotic areas. Tumor samples were further reduced in size using surgical scissors and washed with cold Hanks' Balanced Salt Solution (cat. no. 14025092, Gibco, Thermo Fisher Scientific, Waltham, MA, USA). To 200 mg of sample, 8 mL of enzyme digestion mix was added. The latter was composed of 1 mL of collagenase (20 mg/ml, cat. no. 0130, Sigma Aldrich, St. Louis, MI, USA), 1 mL of hyaluronidase (20 mg/ml, cat. no. H3506, Sigma Aldrich), 2.5 μL of DNAse (100 μg/ul, cat. no. D5025, Sigma Aldrich) in 8 mL of RPMI medium (cat. no. 21875042, Gibco). For further details, see Supplemental Material.

### Cell Sorting

Three to ten million cells were transferred to a clean 15 mL conical tube, while the volume was adjusted to 1 mL using a 0.5% BSA PBS solution. Next, 5 tubes each containing 100,000 cells in 100 μL 0.5% BSA PBS solution were also prepared for individual stains/negative control. The following antibodies/dyes were used to stain the cells (according to the manufacturers' instructions): EPCAM-PE (cat. no. 347198, Becton Dickinson (BD), Franklin Lakes, NJ, USA); CD31-Alexa488 (cat. no. 558068, BD); CD45-APC (cat. no. 560973, BD); and Live-Dead-NearIR (cat. no. L34961, Life Technologies, Thermo Fisher). The antibody-sample mix was incubated for 30 min at RT, then topped with a 0.5% BSA PBS solution to 10 mL, and centrifuged at 300×g for 5 min at 4° C. Cells were suspended in 1 mL of a 0.5% BSA PBS solution and sorted using FACS Aria 2 (BD). For further details, see Supplemental Material.

### Single-Cell RNAseq

As outlined above, samples were processed using the 10× Genomics Single Cell 3′ Reagent Kit v3 (10× Genomics, Pleasanton, CA, USA) user guide. That process can briefly be described as follows: starting with cell suspension, Gel Bead-In Emulsions (GEM) were generated, barcoded, and RT reaction was performed. Purified cDNA was then amplified for 12 cycles, and the resulting cDNA run on a Fragment Analyzer (High Sensitivity kit NGS) (Agilent Technologies, Santa Clara, CA, USA) to determine their quantity. cDNA libraries were then prepared, adjusting the PCR cycles based on the calculated cDNA concentration. For this purpose, Chromium Single Cell 3′ Library and Gel Bead Kit v3, Chromium Single Cell 3′ Chip kit v3, and Chromium i7 Multiplex were used. The proportion of each library was calculated based on preliminary shallow sequencing run using MiniSeq (Illumina, San Diego, CA, USA) and Mid Output Reagent Cartridge (Illumina). After evaluating the number of cells, reads and sequencing saturation, libraries were then pooled in 2 or 3 samples per run and normalized to a final loading concentration. Each run was sequenced on NovaSeq using v1 chemistry. A sequencing depth of 50,000 reads/cell was targeted for each sample. Sequencing fastq files passing Illumina quality control criteria were further analyzed using 10× Genomics CellRanger pipeline v 3.0.2 and 3.1.0.

### Data Preparation and Initial Filtering

Raw data were processed using 10× Genomics Cell Ranger software (v3.0.2). For each sample, the cells with top 0.05% or total UMIs were considered as doublets and therefore removed. Cells with less than 1,000 distinct genes measured were also discarded. Unless specified otherwise, each cell transcriptome was normalized by the total UMI count (division by the total and multiplication by 104), and log-transformed (log 2 (1+norm UMI count)). For further details, refer to Supplemental Material.

### Two-Dimensional Projections and Clustering

EPCAM+ and TN cell 2-dimensional projections were obtained separately, using the 1,500 most variable genes (coefficient of variation) among the 5,000 most expressed genes over the respective populations of cells. We only considered genes expressed in at least 1% of the cells. Principal component analysis (PCA) was computed and the first 30 principal components then submitted to t-SNE (perplexity=30). Clustering of TN cells was achieved by computing Euclidean distances between transcriptomes and constructing a dendrogram using Ward's method. This computation used the same 1,500 genes as the projection.

### Differential Gene Expression Analysis and Gene Signatures

Differentially expressed genes were identified using edgeR.41 For this purpose, TMM normalization was applied by the calcNormFactors function, and glmFit and glmLRT functions were used with default parameters to identify differentially expressed genes. Correction of P-values was obtained following the Benjamini-Hochberg procedure. Signature genes for each of the 4 TN cell clusters (CLU+, C3+, RGS5+, POSTN+) were selected by requiring an expression in at least 20% of the cluster cells, a fold-change (FC)≥2 and an adjusted P-value≤1% in a comparison against the 3 other clusters pooled together, or in the 3 comparisons against each cluster separately. The top 30 such genes were kept, sorted according to FC. The signature genes for the 2 main TN clusters (MCAM+ and LTBP2+) were obtained requiring expression in at least 20% of the main cluster cells, and in at least 10% of each of the subcluster cells (e.g., in CLU+ and RGS5+ for MCAM+), a FC≥2 and an adjusted P≤1% comparing with the other main cluster (MCAM+ versus LTBP2+) or with its subclusters (e.g. MCAM+ versus C3+ or POSTN+). We kept the top 30 such genes sorted according to FC.

### Non-Cancer Mesenchymal Liver Cells

Mesenchymal single-cell transcriptomes in the liver were retrieved from a published atlas covering four healthy and three cirrhotic human livers 13. Among these cells, the authors identified four clusters, Mes(1), Mes(2), Mes(3), and Mes(4). Mes(4) was discarded in our study, since it was identified as mesothelial cells. We constructed Mes(1-3) gene signatures following the same procedure as above: required expression in at least 20% of the cells of a given cluster, a FC≥2 and an adjusted P≤1% when comparing with each other cluster, or with union of the other two clusters. Less genes satisfied such criteria in this dataset, and we therefore limited the size of the signatures to the top 16 genes according to FC.

### Machine Learning

We built three different models to classify new cells to belong to one of Ramachandran, et al., Mes(1), Mes(2), or Mes(3) mesenchymal cell subtypes 13. We used random forest (R randomForest package, default parameters), K nearest neighbors (R DMwR package, function kNN, k=100, norm=FALSE), and support vector machine (R package caret; trainControl with method=“repeatedcv”, number-10, repeats=3; train with method= “svmLinear”, preprocess=c (“center”, “scale”), tuneLength=10). Algorithm performance was evaluated by cross-validation using 90% of the cells to train the models and 10% to test them, repeated 20 times.

### Cell Culture

The HT29, LOVO and CCD18Co cells were obtained from ATCC (Virginia, USA). SW1222 cells were a kind gift by Prof. W. Bodmer, Department of Medical Oncology, Weatherall Institute, Oxford, UK. A CAF cell line was isolated from a CRC-LM. LX2? CCD18Co cells were immortalized using. All cell lines were cultured in Dulbecco's Modified Eagle Medium (DMEM) supplemented with 10% fetal bovine serum (FBS) and 1% penicillin/streptomycin (all from Gibco, Thermo Fisher Sci., Waltham, MA, USA) at 37° C. in 5% CO2.

Conditioned medium (CM) from CRC cell lines was obtained after 48 h incubation of 80% confluent cells in serum-free DMEM. CM were collected, centrifuged at 150×g for 5 min at RT, and then added to CCD18Co, LX2 and CAF cell monolayers (cells were pre-starved in serum-free medium for 6h) for 48 h. The control consists in the addition of serum-free DMEM. Then, CM medium were collected for western blot analysis and processed in the same way as previously. The cell monolayers were washed with PBS twice and lysed for RNA extraction.

Human anti-LTBP2 siRNA (ON-TARGET plus Human LTBP2 (4053), catalog no. L-011078-00-0005) and control siRNA (ON-TARGET plus Non-targeting Pool, catalog no. D-001810-10-5) were from Dharmacon (Lafayette, USA). CCD18Co cells were transfected with 40 nM of siRNA using Lipofectamine (Lipofectamine 2000 reagent, catalog no. 11668-019, Life Technologies, Carlsbad, CA, USA). After 48h, cell monolayers were washed with PBS twice and lysed for RNA extraction.

Phage-display selection of LTBP2 antibodies His-tagged LTBP2 was produced by cloning the LTBP2 ORF (Cat. No.: OHu107637, GeneScrip, Piscataway, NJ, USA) in the pCMV vector (Cat. No.: 212220, Agilent, Santa Clara, CA, USA) and by transient transfection in HEK293 cells. Following HEK293 culture in standard conditions, the media were collected, centrifuged and the recombinant LTBP2 was purified using Ni-chromatography (Cat. No.: A50585, Thermo Fisher Sci., Waltham, MA, USA). Phage display selection of anti-LTBP2 antibodies was performed by the Montpellier Biocampus academic platform GenAc.

Cell Culture Treatment with Antibodies and MTT Assay

CCDCl8Co cells were cultured in DMEM under standard conditions while cancer cells conditioned media were derived as described above. For the experiment, the conditioned media were diluted 1:1 with fresh DMEM and 1% FBS was added to the mix. Following this, the mixture was transferred to the CCD18Co cells while antibodies were also added at 5 ug/mL concentration. The cells were then incubated under standard cell culture conditions for 120h. Following this, the cell viability was assessed using 3-(4,5-dimethyl thiazol-2-yl) 2,5-diphenyl tetrazolium bromide) (MTT) staining (catalog no. M5655, Sigma-Aldrich). Absorbance was measured at 540 nm.

### Western Blot Analysis

CM were concentrated 10-fold using vivaspin columns 10 kDa filters (catalog no. VS0102, Sartorius stedim biotech, Stonehouse, UK). The cell culture medium was exchanged with the RIPA buffer (150 mM NaCl, 0.5% Na-deoxycholate, 1% Triton X-100, 0.5% SDS, 50 mM Tris-HCl (pH 7.5)). Laemmli buffer (0.1% 2-mercaptoethanol, 0.0005% bromophenol blue, 10% glycerol, 2% SDS in 63 mM Tris-HCl (pH 6.8)) was added to 20 μl of concentrated CM. CM were then boiled 5 min and loaded on 6% polyacrylamide gels. Proteins were transferred to nitrocellulose membranes at 100V for 2 h. After blocking 1 h in 5% skim milk, membranes were incubated (4° C., overnight) with antibody against LTBP2 (1:500; catalog no. AF3850, R&D systems, Minneapolis, USA).

### Silver Staining

Laemmli buffer was added to five microliters of 10× concentrated CM. After boiling the samples 5 min, they were loaded on 10% polyacrylamide gels. Gels were stained using PlusOne Silver Staining Kit, Protein (catalog no. 17-1150-01, GE Healthcare, Uppsala, USA).

### Gene Expression Analysis by Real Time Quantitative PCR (RT-qPR)

Total RNA was isolated with Monarch Total RNA Miniprep Kit (catalo no. T2010S, New England Biolabs). For RT-qPRC analysis, RNA was reverse-transcribed using the SuperScript III Reverse Transcriptase (catalog no. 18080; Invitrogen, Carlsbad, CA, USA). Twenty nanograms of cDNA were used for respective PCR reactions.

The comparison of basal level gene expression has been performed on 3 biological replicates for the CAF and LX2 and 4 replicates for CCD18Co. Cp values were compared using an unpaired Welch's test thanks to the t.test R function (var.equal=F).

The evaluation of CRC cells CM effect has been performed on 2, 3 and 4 biological replicates for the CAF, LX2 and CCD18CohTert respectively. For each cell line, ΔCp values were compared between two conditions with a paired Student's test (var.equal=F). Fold changes in comparison to the control condition are reported.

### Bulk RNA Sequencing

Total RNA was isolated as previously described. For each sample, 1 μg of total RNA was used to construct the sequencing libraries. Libraries were prepared with the RNA Stranded Total RNA prep Ligation with Ribo-Zero plus kit (Illumina, San Diego, USA) in order to deplete ribosomal RNA. Then, they were sequenced on a NovaSeq6000 SP-200 cycles (Illumina) to generate 66 million reads in each direction per sample. Basecalling and demultiplexing steps were performed using Illumina software Dragen 3.8.4. With our pipeline, Fastq files were aligned against the human genome (Ensembl GRCh38) (STAR using default parameters and 2 passes, read counts extraction with HTSeq-count).

TMM normalization was applied by the calcNormFactors function (edgeR R package), and glmFit and glmLRT functions were used with default parameters to identify differentially expressed genes. Correction of P-values was obtained following the Benjamini-Hochberg procedure (multitest R package). Normalized transcriptomes were then log-transformed (x□log2(x+1)), and z-scores were computed.

Functional analysis of the differentially expressed genes (adjusted p-value<0.01, fold change absolute value>2) has been realized by performing hypergeometric tests on Gene Ontology biological processes (GOBP) containing at least 3 differentially expressed genes.

### Data Access and Sample IDs

Single-cell transcriptomes are available from GEO with reference GSE158692. In these data, patient 1 metastasis (P1_MP) is referenced as SC_196081, P2_MP as 19G00619, P3_MP as 19G00635, patient 4 metastasis a (P4_MPa) as 19G02977_Big, and b (P4_MPb) as 19G02977_Small, and P5_MP as 20G00953. Cell culture RNA-sequencing data are available from GEO with reference GSE191323.

### Results

### CRC-LM CAFs are Comprised of Distinct Subpopulations

In this study, we isolated CAFs from metastases based on a triple negative selection strategy (EPCAM−/CD45−/CD31−/LiveDead−). This strategy was chosen because of the absence of universal CAF cell-surface markers and the potential heterogeneity of this cell population. Other recent single-cell CAF studies have followed a similar procedure (7,8). After data quality filtering and the elimination of a few contaminating cells (n=215, mainly hepatocytes, data not shown), we obtained the individual transcriptomes of a total of 4,397 CAFs (data not shown). These CAFs clustered into two major groups, indicating the existence of two main populations of CAFs (data not shown). A second level of clustering decomposed the two main populations into four more specialized CAF clusters (data not shown). Differential gene expression analysis identified population-specific genes at the two levels of decomposition (data not shown). Normalized expression levels of six representative genes was done, along with the dendrogram of complete CAF transcriptomes and led to the identification of the four CAF populations. Each metastasis harbored all of the CAF populations, but P3_MP that was devoid of CLU+ (data not shown).

Every CAF population harbored genes underlying typical CAF functions, such as an important production of collagen and ECM-related components (data not shown). Nonetheless, there were significantly different levels of activity in several GO biological processes across those populations (data not shown). LTBP2+ CAFs were more involved in ECM remodeling (data not shown) and collagen production (data not shown). FAP, a marker of fibroblast activation and proliferation, and PDGFRA, a marker of connective tissue remodeling 18, were also expressed by LTBP2+ CAFs specifically (data not shown). LTBP2+/POSTN+ CAFs, which represent the majority of LTBP2+ CAFs, were even more active in a number of areas. These included: collagen production (data not shown), TGF-β response as illustrated by POSTN and INHBA expression (data not shown), angiogenesis (VEGFC, and UNC5B and SRPX2, two known angiogenesis-involved genes (19,20) data not shown), and Wnt signaling. LTBP2+/C3+ CAFs expressed complement genes (e.g., C7 and CFD, data not shown), but also CLU at an intermediary level (data not shown). Complement genes are known to have a potential immunosuppressive effect in certain tumors, including CRC, in particular when regulators of the complement cascade such as CLU are coexpressed (21). MCAM+ CAFs expressed markers of blood vessel wall such as RGS5, a known pericyte gene. MCAM itself is a known pericyte and VSMC gene. MYH11 expression was also strong in MCAM+ CAFs; it is a marker of contractility. In agreement with their higher muscle contraction signature, MCAM+/CLU+ CAFs expressed additional markers of contractility such as PLN and ACTG2 (data not shown), but did not do so exclusively. Moreover, MCAM+/RGS5+ CAFs displayed an average CAF phenotype (data not shown), which resulted in no specific enrichment (data not shown). A recent report discussing TME heterogeneity across multiple cancers 15 identified five commonly found CAF populations. We matched our CAF gene signatures with these data and found highly significant overlaps (data not shown). We also obtained significant overlap of the LTBP2+ gene signature with a CAF population (CAF-S1), enriched in triple negative breast tumors 7 (data not shown).

Attempting to explain the heterogeneity of phenotypes observed in the identified CRC-LM CAF populations, we compared our data with an atlas that portrayed healthy and cirrhotic liver cell types (13). As explained in Introduction, we identified three populations of relevant mesenchymal cells in the liver: VSMCs, HSCs, and cells strongly associated with fibrosis that expressed PDGFRA, but were devoid of RGS5 expression. These latter cells are named SAMes (scar-associated mesenchymal cells). Employing the same procedure as above, we computed gene signatures for these three populations (data not shown). Remarkably, the significant intersection of our four CAF populations with those signatures indicated phenotypic proximity between VSMCs and MCAM+/CLU+ CAFs, HSCs and MCAM+/RGS5+ CAFs, and SAMes and LTBP2+ CAFs (data not shown). Following a machine learning (ML) approach, we tested this potential association. We first constructed and evaluated different classifiers (random forest, support vector machine, and k-nearest neighbors) based on the healthy/cirrhotic liver single-cell data (13). Standard cross-validation, where 90% of the data were used to train the models and 10% to test them, resulted in good performance estimations (data not shown). We therefore applied these classifiers to our CRC-LM CAF transcriptomes and indeed confirmed the association pattern (data not shown). Remarkably, a comparison of the CAFs with their matched non-cancer mesenchymal cells showed a near-systematic significant increase of the processes reported, compared to the non-cancer situation (data not shown). Lastly, we interrogated CAF single-cell transcriptomes of hepatocellular carcinoma (HCC) and intrahepatic cholangiocarcinoma (iCCA) (22), aiming to assess the presence of CAFs similar to our CRC-LM LTBP2+ and MCAM+ CAFs. Strikingly, while these two CAF populations featured sizes in iCCA comparable to our data in CRC-LM, the LTBP2+ CAFs were strongly depleted in HCC (data not shown).

We confirmed the expression of the three reporter genes related to LTBP2+ CAFs (LTBP2, C3, and POSTN) by RT-qPCR in relevant mesenchymal cells, upon their exposure to conditioned media of CRC cancer cells. Namely, we employed a stellate cell line (LX2), fibroblasts (CCD18Co) and patient CRC-LM CAFs. Basal expression levels were obtained in cells incubated in DMEM (data not shown). We found LTBP2 and POSTN to be overexpressed in CAFs compared to LX2 and CCD18Co cells, while the opposite pattern was true for C3. This suggested that the CAFs obtained by the patient at hand were of the LTBP2+/POSTN+ subtype. Furthermore, LX2 stellate cells tended to express less of LTBP2+ gene markers (LTBP2, C3, and POSTN), which is compatible to the stellate cell origin of RGS5+ CAFs in our single-cell data. To compare to the situation where mesenchymal cells are exposed to nearby cancer cells, we incubated LX2 and CCD18Co cells, and patient-derived CAFs with the conditioned media of three different CRC cell lines. We observed that only fibroblast CCD18Co cells increased C3 and POSTN expression (data not shown). Absence of induction in CAFs might be due to the very high basal levels, whereas LX2 stellate cells were unable to respond to the challenge for those two genes. Regarding LTBP2, we found a modest trend suggesting that main regulation could rather operate at the protein level. Indeed, Western blots on the conditioned media of those cells that were used for RT-qPCR confirmed this hypothesis (data not shown).

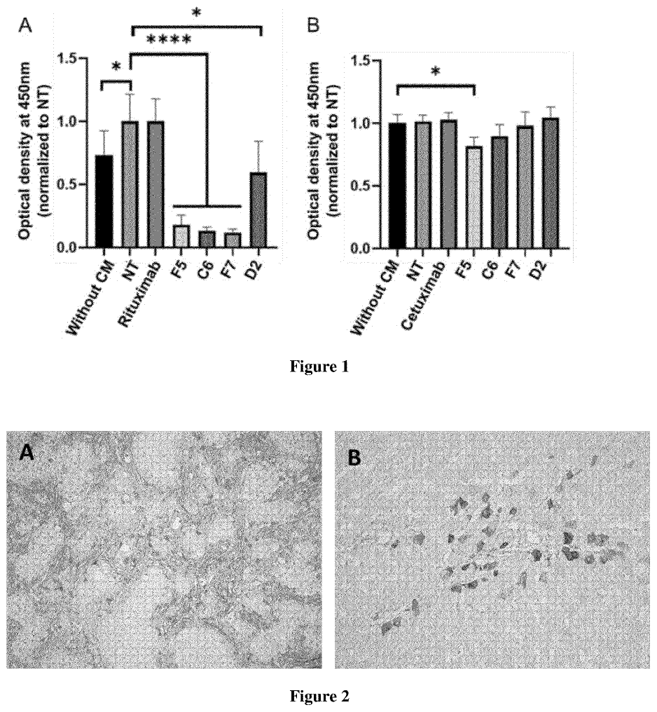

LTBP2 is a secreted protein that bares the potential to be systemically reachable and targetable in vivo. Guided by this aspect, we employed phage-display technology to select a panel of four fully human IgG anti-LTBP2 antibodies. Treatment of fibroblasts (CCD18Co cells) with the panel of anti-LTBP2 antibodies reduced their viability (as judged by the MTT assay) significantly (FIG. 1A), but not HT29 CRC cells (FIG. 1B) indicating specificity for fibroblasts. Closer investigation showed that 96 h post anti-LTBP2 treatment, the fibroblasts became round and started detaching from the well. Propidium iodide/Hoechst staining of the detached cells revealed no apparent apoptosis or necrosis (data not shown) suggesting a more subtle LTBP2-mediated mechanism of action. To obtain information about the functional importance of LTBP2 for fibroblast biology, we silenced its gene expression by siRNAs and performed RNA-sequencing. We found as many as 496 significantly deregulated genes suggesting an important role (data not shown). Roughly half the deregulated genes featured increased expression upon LTBP2 silencing, while the other half decreased. GO biological process enrichment analysis identified several deregulated pathways. Representative genes of some pathways such as integrins, collagens, LOX (involved in ECM collagen cross-linking and stiffness), or CD151 (involved in cell adhesion) have been identified.

### LTBP2+ Fibroblasts are Found in Portal Spaces of the Normal Human Liver

SAMes globally expressed portal fibroblast markers, and a subpopulation of SAMes cells was located at the periportal space 13. Our analysis above showed LTBP2+ CAFs relation to SAMes. We thus sought to validate their origin in the adjacent normal human liver. Triple immunofluorescence staining of pan-cytokeratin (epithelial compartment; data not shown), α-SMA (stellate cells/fibroblasts) and LTBP2, revealed a distinct enrichment of LTBP2+ cells in the portal regions of normal liver (data not shown). In a further investigation, LTBP2 staining coincided with typical aspects of collagen-containing connective tissue found in the portal space. LTBP2-expressing cells were also positive for α-SMA, although the two proteins did not co-localize. This was not surprising, as α-SMA is known to be a cytoskeletal protein, while LTBP2 is mainly secreted. α-SMA was especially positive in the Disse space, labelling stellate cells. No notable LTBP2 staining, however, was visible in the Disse space or hepatocytes. In the tumoral tissue, double stained LTBP2+/α-SMA+ CAFs (data not shown) were clearly distinguishable from CAFs expressing α-SMA+ only (data not shown). For reasons of simplicity, we choose to further distinguish only LTBP2+ and α-SMA+ CAFs. We assume that LTBP2+ CAFs are also positive for α-SMA+.

LTBP2+ CAFs Accumulate at Tumor Locations with Strong Desmoplastic Reaction

Bioinformatic analysis above indicated that LTBP2+ CAFs were significantly implicated in ECM remodeling. Further analysis revealed that LTBP2+ CAFs tended to accumulate at areas of strong desmoplastic reaction. To confirm this initial observation, we exploited the invasive fronts a cohort of 20 liver metastases that were classified based on their so-called histologic growth patterns (HGPs) (23-25). This classification evaluates the transition zone where cancer cells grow towards normal liver parenchyma, the surrounding stromal cells, and the extracellular matrix (ECM). Three patterns are defined: The desmoplastic (or encapsulated) HGP is characterized by extensive collagen deposition, prominent angiogenesis, and no contact between tumor cells and the hepatocytes. The pushing (or expansive) HGP is devoid of desmoplastic reaction, tumor cells are separated from hepatocytes by a thin reticulin fiber layer, and liver cells are pushed away by the metastasis. A mild immune infiltrate can be present at the interface. In the replacement HGP, cancer cells infiltrate the liver parenchyma without any disturbance of its structure, contrary to the other two HGPs. There is no fibrosis and barely any inflammation. Some metastases might display distinct HGPs depending on the location. Analysis of immunofluorescence images clearly showed that the proportion of LTBP2+ CAFs was significantly higher at the invasive front of tumors displaying the desmoplastic HGP, compared to the replacement or pushing patterns (data not shown). Cases involving mixed growth patterns (two distinct regions presenting different HGPs) were also included and accounted at the local level separately.

Independent of the invasive front and the HGP, metastases harboring a strong desmoplastic reaction at their center also contained an increased proportion of LTBP2+ CAFs (data not shown).

### LTBP2+ CAF and Angiogenesis

In addition to ECM remodeling, bioinformatics indicated an increased activity of angiogenesis-related pathways in LTBP2+ CAFs. We hence tested whether angiogenesis was also correlated with the presence of LTBP2+ CAFs. To this end, we evaluated CD31 positivity along with LTBP2 staining in 20 CRC-LMs. Normal/cancer interfaces of replacement and pushing patterns provided LTBP2+ CAF poor areas, where immunofluorescence analysis evidenced small, capillary-type vessels (data not shown), similar to those observed in normal hepatic parenchyma. This was in strong contrast with highly desmoplastic areas, where numerous large vessels (not capillaries) were readily observable (data not shown). The latter is consistent with existing reports which underscore the importance of de novo angiogenesis in desmoplastic HGP (26). Similar to ECM remodeling, angiogenesis also associates with LTBPT2+ CAF density at the center of metastases when concomitant with a strong desmoplastic reaction (data not shown). LTBP2 expression positively correlated with vessel size across all cases (data not shown).

### LTBP2+ CAFs and Cancer Cell Growth

Areas where LTBP2+ CAFs were present in high quantities displayed a prominent desmoplastic reaction. While we did not specifically stain for collagen, hematoxylin/eosin images as well as scRNAseq data clearly indicated significant collagen deposition/production by LTBP2+ CAFs. Collagen leads to a more abundant (and certainly stiffer) ECM. We thus hypothesized that this mechanical constraint would lower the growth ability of cancer cells. To verify this, we examined the CRC-LM cases for Ki67 expression, which showed that Ki67 positivity in cancer cells negatively correlated with LTBP2+ CAF abundance (data not shown).

### Mapping CAFs/Cancer Cells Cellular Interactions

The EPCAM+ cells obtained from the six CRC-LMs (5,331 cells in total) formed well-defined clusters correlating with the patient of origin (data not shown). Notably, the two metastases from patient 4 grouped together indicating modest transcriptional divergence. To infer ligand-receptor (LR) interactions between cancer cells and the two main populations of CAFs (MCAM+ and LTBP2+), we employed SingleCellSignal®, a Bioconductor package which we published recently (27). Inferences rely on a curated database of known in vivo and in vitro LR interactions and the computation of a score for each interaction, the so-called LR-score. An LR-score≥0.5 is sufficient to trust the interaction (27). For LR interactions between cancer cells and CAFs, we computed six LR-scores, one per metastasis, and imposed the 0.5 threshold on the median LR-score (in general, LR-scores of distinct metastases were close to each other, data not shown). We found that the highest number of paracrine LR interactions occurred between CAFs, followed by CAF-to-cancer cell interactions, and cancer cell-to-CAF interactions (data not shown). Most of the molecules involved in these interactions were growth factors or related to the ECM, cell-cell interactions, or chemotaxis.

Next, we focused on the difference between interactions linking LTBP2+ or MCAM+ CAFs to cancer cells. Based on median LR-score differences (|med.MCAM.LRscore-med.LTBP2.LRscore|>0.1) and CAF-secreted ligand differential gene expression (FDR<1%, FC>2) between MCAM+ and LTBP2+ CAFs, we could call LR interactions with a significant bias in strength (data not shown). We identified 178 significantly stronger interactions from LTBP2+ CAFs to cancer cells (data not shown), and 14 significantly stronger from MCAM+ (data not shown). Stronger interactions originating from LTBP2+ CAFs included growth factors, Wnt signaling, and angiogenic interaction. In addition, MCAM+ and LTBP2+ CAFs seemed to modulate laminin trimers differently. Selection of significantly biased LR interactions relating cancer cells to the two CAF populations identified 81 stronger cases towards LTBP2+ CAFs and 13 towards MCAM+ CAFs (data not shown). The larger numbers of significantly stronger interactions with LTBP2+ CAFs, in both directions, reflect their higher propensity to act with a more specific phenotype, as previously suggested (data not shown).

### Canonical Wnt Signaling is Upregulated in Cancer Cells Found Near LTBP2+ CAFs

Cellular interaction inference suggested that LTBP2+ CAFs were inducting Wnt/β-catenin signaling in cancer cells (data not shown). Knowing the importance of Wnt in CRC we sought to validate this observation at the protein level. We co-stained LTBP2 and β-catenin in CRC-LMs and evaluated the extent of nuclear β-catenin staining near LTBP2+ CAFs. Nuclear accumulation of β-catenin was observable in cancer cells located in LTBP2+ CAF-rich areas. A correlation analysis confirmed significant association between the two proteins (data not shown).

### Comparing CAF Cells of Origin in CRC-LM and Other Liver Malignancies

Published single-cell data on iCCA and HCC 22 already enabled us to show the existence of CAF populations baring the LTBP2+ and MCAM+ gene signatures (data not shown). The application of our ML model indicated that both iCCA and CRC-LM harbor comparable and significant proportions of portal fibroblast-derived CAFs (data not shown). On the contrary, HCC harbors HSC-derived CAFs in majority (data not shown).

### Cancer Cells May Adopt CAF Features

Surprisingly, the inventors showed that LTBP2 may be expressed in a few cancer cells, in particular aggressive cancer cells (FIG. 2).

### LTBP2 Enhances HCC Invasion In Vitro

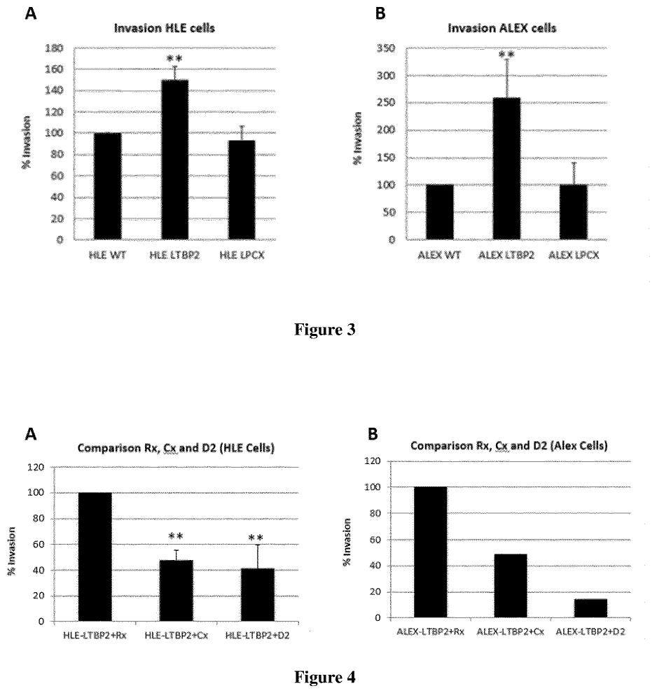

The percentage of HCC invasion (HLE cell and ALEX cell) is higher in HCC cells engineered to express LTBP2 than other groups (FIG. 3). Thus, the cells expressing LTBP2 are strongly involved in cancer progression and metastasis. Moreover, LTBP2 is a functional target promoting tumor development in human HCC.

### LTBP2 Inhibitors Stop the Invasion of HCC Expressing LTBP2

Anti-LTBP2 inhibitors and particularly the antibodies of the invention, reduce the invasion of HCC cells expressing LTBP2. The percentage of HCC invasion is better reduced with D2 (FIG. 4) notably compared to Rituximab and Cetuximab

### REFERENCES

Throughout this application, various references describe the state of the art to which this invention pertains. The disclosures of these references are hereby incorporated by reference into the present disclosure.
1 Dekker E, Tanis P J, Vleugels J L A, Kasi P M, Wallace M B. Colorectal cancer. Lancet 2019; 394:1467-1480.
2 Aranda E, Aparicio J, Alonso V, Garcia-Albeniz X, Garcia-Alfonso P, Salazar R et al. SEOM clinical guidelines for diagnosis and treatment of metastatic colorectal cancer 2015. Clin Transl Oncol 2015; 17:972-981.
3 Junttila M R, de Sauvage F J. Influence of tumour micro-environment heterogeneity on therapeutic response. Nature 2013; 501:346-354.
4 Emambux S, Tachon G, Junca A, Tougeron D. Results and challenges of immune checkpoint inhibitors in colorectal cancer. Expert Opin Biol Ther 2018; 18:561-573.
5 Ronca R, Van Ginderachter J A, Turtoi A. Paracrine interactions of cancer-associated fibroblasts, macrophages and endothelial cells: tumor allies and foes. Current opinion in oncology. 2018; 30. doi:10.1097/CCO.0000000000000420.
6 Chiavarina B, Turtoi A. Collaborative and Defensive Fibroblasts in Tumor Progression and Therapy Resistance. Current medicinal chemistry. 2017; 24. doi:10.2174/0929867324666170428104311.
7 Kieffer Y, Hocine H R, Gentric G, Pelon F, Bernard C, Bourachot B et al. Single-Cell Analysis Reveals Fibroblast Clusters Linked to Immunotherapy Resistance in Cancer. Cancer Discov 2020; 10:1330-1351.
8 Costa A, Kieffer Y, Scholer-Dahirel A, Pelon F, Bourachot B, Cardon M et al. Fibroblast Heterogeneity and Immunosuppressive Environment in Human Breast Cancer. Cancer Cell 2018; 33:463-479.e10.
9 LeBleu V S, Kalluri R. A peek into cancer-associated fibroblasts: origins, functions and translational impact. Disease Models & Mechanisms 2018; 11. doi:10.1242/dmm.029447.
10 Baglieri J, Brenner D A, Kisseleva T. The Role of Fibrosis and Liver-Associated Fibroblasts in the Pathogenesis of Hepatocellular Carcinoma. International Journal of Molecular Sciences 2019; 20:1723.
11 Seki E, Schwabe R F. Hepatic inflammation and fibrosis: Functional links and key pathways. Hepatology 2015; 61:1066-1079.
12 Mederacke I, Hsu C C, Troeger J S, Huebener P, Mu X, Dapito D H et al. Fate tracing reveals hepatic stellate cells as dominant contributors to liver fibrosis independent of its aetiology. Nature Communications 2013; 4:2823.
13 Ramachandran P, Dobie R, Wilson-Kanamori J R, Dora E F, Henderson BEP, Luu N T et al. Resolving the fibrotic niche of human liver cirrhosis at single-cell level. Nature 2019; 575:512-518.
14 Kim N, Kim H K, Lee K, Hong Y, Cho J H, Choi J W et al. Single-cell RNA sequencing demonstrates the molecular and cellular reprogramming of metastatic lung adenocarcinoma. Nat Commun 2020; 11:2285.
15 Qian J, Olbrecht S, Boeckx B, Vos H, Laoui D, Etlioglu E et al. A pan-cancer blueprint of the heterogeneous tumor microenvironment revealed by single-cell profiling. Cell Res 2020; 30:745-762.
16 Che L-H, Liu J-W, Huo J-P, Luo R, Xu R-M, He C et al. A single-cell atlas of liver metastases of colorectal cancer reveals reprogramming of the tumor microenvironment in response to preoperative chemotherapy. Cell Discov 2021; 7:80.
17 Massalha H, Bahar Halpern K, Abu-Gazala S, Jana T, Massasa E E, Moor A E et al. A single cell atlas of the human liver tumor microenvironment. Mol Syst Biol 2020; 16: e9682.
18 Horikawa S, Ishii Y, Hamashima T, Yamamoto S, Mori H, Fujimori T et al. PDGFRα plays a crucial role in connective tissue remodeling. Scientific Reports 2015; 5:17948.
19 Miljkovic-Licina M, Hammel P, Garrido-Urbani S, Bradfield P F, Szepetowski P, Imhof B A. Sushi repeat protein X-linked 2, a novel mediator of angiogenesis. FASEB J 2009; 23:4105-4116.
20 Navankasattusas S, Whitehead K J, Suli A, Sorensen L K, Lim A H, Zhao J et al. The netrin receptor UNC5B promotes angiogenesis in specific vascular beds. Development 2008; 135:659-667.
21 Roumenina L T, Daugan M V, Petitprez F, Sautès-Fridman C, Fridman W H. Context-dependent roles of complement in cancer. Nature Reviews Cancer 2019; 19:698-715.
22 Ma L, Hernandez M O, Zhao Y, Mehta M, Tran B, Kelly M et al. Tumor Cell Biodiversity Drives Microenvironmental Reprogramming in Liver Cancer. Cancer Cell 2019; 36:418-430.e6.
23 Vermeulen P B, Colpaert C, Salgado R, Royers R, Hellemans H, Van Den Heuvel E et al. Liver metastases from colorectal adenocarcinomas grow in three patterns with different angiogenesis and desmoplasia. J Pathol 2001; 195:336-342.
24 Stessels F, Van den Eynden G, Van der Auwera I, Salgado R, Van den Heuvel E, Harris A L et al. Breast adenocarcinoma liver metastases, in contrast to colorectal cancer liver metastases, display a non-angiogenic growth pattern that preserves the stroma and lacks hypoxia. Br J Cancer 2004; 90:1429-1436.
25 Van den Eynden G G, Majeed A W, Illemann M, Vermeulen P B, Bird N C, Høyer-Hansen G et al. The multifaceted role of the microenvironment in liver metastasis: biology and clinical implications. Cancer Res 2013; 73:2031-2043.
26 Frentzas S, Simoneau E, Bridgeman V L, Vermeulen P B, Foo S, Kostaras E et al. Vessel co-option mediates resistance to anti-angiogenic therapy in liver metastases. Nature Medicine 2016; 22:1294-1302.
27 Cabello-Aguilar S, Alame M, Kon-Sun-Tack F, Fau C, Lacroix M, Colinge J. SingleCellSignalR: inference of intercellular networks from single-cell transcriptomics. Nucleic Acids Res 2020. doi:10.1093/nar/gkaa 183.

1. An antibody having a heavy chain comprising i) an H-CDR1 of C6 mab, ii) an H-CDR2 of C6 mab and iii) an H-CDR3 of C6 mab and a light chain comprising i) an L-CDR1 of C6 mab, ii) an L-CDR2 of C6 mab and iii) an L-CDR3 of C6 mab
wherein
the H-CDR1 of the C6 mab is defined by the sequence ranging from the amino acid residue at position 31 to the amino acid residue at position 35 in SEQ ID NO: 1;
the H-CDR2 of C6 mab is defined by the sequence ranging from the amino acid residue at position 50 to the amino acid residue at position 66 in SEQ ID NO:1;
the H-CDR3 of the C6 mab is defined by the sequence ranging from the amino acid residue at position 100 to the amino acid residue at position 113 in SEQ ID NO: 1;
the L-CDR1 of the C6 mab is defined by the sequence ranging from the amino acid residue at position 23 to the amino acid residue at position 36 in SEQ ID NO: 2;
the L-CDR2 of the C6 mab is defined by the sequence ranging from the amino acid residue at position 52 to the amino acid residue at position 58 in SEQ ID NO: 2;
the L-CDR3 of the C6 mab is defined by the sequence ranging from the amino acid residue at position 91 to the amino acid residue at position 100 in SEQ ID NO: 2;
or an antibody having a heavy chain comprising i) an H-CDR1 of D2 mab, ii) an H-CDR2 of D2 mab and iii) an H-CDR3 of D2 mab and a light chain comprising i) an L-CDR1 of D2 mab, ii) an L-CDR2 of D2 mab and iii) the L-CDR3 of D2 mab
wherein
the H-CDR1 of the D2 mab is defined by the sequence ranging from the amino acid residue at position 31 to the amino acid residue at position 35 in SEQ ID NO: 1;
the H-CDR2 of D2 mab is defined by the sequence ranging from the amino acid residue at position 50 to the amino acid residue at position 66 in SEQ ID NO:1;
the H-CDR3 of the D2 mab is defined by the sequence ranging from the amino acid residue at position 100 to the amino acid residue at position 108 in SEQ ID NO: 1;
the L-CDR1 of the D2 mab is defined by the sequence ranging from the amino acid residue at position 23 to the amino acid residue at position 36 in SEQ ID NO: 2;
the L-CDR2 of the D2 mab is defined by the sequence ranging from the amino acid residue at position 52 to the amino acid residue at position 58 in SEQ ID NO: 2;
the L-CDR3 of the D2 mab is defined by the sequence ranging from the amino acid residue at position 91 to the amino acid residue at position 100 in SEQ ID NO: 2;
or antibody having a heavy chain comprising i) an H-CDR1 of F5 mab, ii) an H-CDR2 of F5 mab and iii) an H-CDR3 of F5 mab and a light chain comprising i) an L-CDR1 of F5 mab, ii) th an e L-CDR2 of F5 mab and iii) an L-CDR3 of F5 mab
wherein
the H-CDR1 of the F5 mab is defined by the sequence ranging from the amino acid residue at position 31 to the amino acid residue at position 35 in SEQ ID NO: 1;
the H-CDR2 of F5 mab is defined by the sequence ranging from the amino acid residue at position 50 to the amino acid residue at position 66 in SEQ ID NO:1;
the H-CDR3 of the F5 mab is defined by the sequence ranging from the amino acid residue at position 99 to the amino acid residue at position 107 in SEQ ID NO: 1;
the L-CDR1 of the F5 mab is defined by the sequence ranging from the amino acid residue at position 23 to the amino acid residue at position 36 in SEQ ID NO: 2;
the L-CDR2 of the F5 mab is defined by the sequence ranging from the amino acid residue at position 52 to the amino acid residue at position 58 in SEQ ID NO: 2;
the L-CDR3 of the F5 mab is defined by the sequence ranging from the amino acid residue at position 91 to the amino acid residue at position 100 in SEQ ID NO: 2;
or an antibody having a heavy chain comprising i) an H-CDR1 of F7 mab, ii) an H-CDR2 of F7 mab and iii) an H-CDR3 of F7 mab and a light chain comprising i) an L-CDR1 of F7 mab, ii) an L-CDR2 of F7 mab and iii) an L-CDR3 of F7 mab
wherein
the H-CDR1 of the F7 mab is defined by the sequence ranging from the amino acid residue at position 31 to the amino acid residue at position 35 in SEQ ID NO: 1;
the H-CDR2 of F7 mab is defined by the sequence ranging from the amino acid residue at position 50 to the amino acid residue at position 66 in SEQ ID NO:1;
the H-CDR3 of the F7 mab is defined by the sequence ranging from the amino acid residue at position 99 to the amino acid residue at position 109 in SEQ ID NO: 1;
the L-CDR1 of the F7 mab is defined by the sequence ranging from the amino acid residue at position 23 to the amino acid residue at position 36 in SEQ ID NO: 2;
the L-CDR2 of the F7 mab is defined by the sequence ranging from the amino acid residue at position 52 to the amino acid residue at position 58 in SEQ ID NO: 2;
the L-CDR3 of the F7 mab is defined by the sequence ranging from the amino acid residue at position 91 to the amino acid residue at position 100 in SEQ ID NO: 2.

2. The antibody according to claim 1 having a heavy chain having at least 70% of identity with SEQ ID NO:1, 3, 5 or 7 and a light chain having at least 70% identity with SEQ ID NO:2, 4, 6 or 8.

3. The antibody according to claim 2 having a heavy chain identical to SEQ ID NO:1, 3, 4 or 7 and a light chain identical to SEQ ID NO: 2, 4, 6 or 7.

4. The antibody of claim 1 which is a chimeric antibody.

5. The antibody of claim 1 which is a humanized antibody which comprises the CDRs of the C6, D2, F5 or F7 mab antibodies.

6. A nucleic acid molecules encoding the antibody of claim 1.

7. The antibody of claim 1 which is conjugated to a cytotoxic moiety.

8. (canceled)

9. A method of treating cancer in a subject in need thereof comprising administering to the subject a therapeutically effective amount of at least one antibody of claim 1.

10. The method of claim 9 wherein the cancer is a colorectal cancer, a liver cancer, a pancreatic cancer, a breast cancer or an associated liver metastases.

11. A method of treating a cancer linked to cancer-associated fibroblasts (CAF) in a subject in need thereof comprising administering to the subject a therapeutically effective amount of at least one antibody of claim 1.

12. A pharmaceutical composition which comprises a pharmaceutically acceptable carrier and at least one antibody according to claim 1.


SEQUENCE LISTING (8 sequences)

SEQ ID NO: 1 - protein, 123 aa, synthetic construct
EVQLVESGGSLVKPGGSLRLSCAASGFTFSNYGMNWVRQAPGKGLEWISSIYGSSRYISYADFVKGRFTISRDNATNSLYLQMNSLRAEDTAVYYCVRSSSYNSYYGGGMDVWGRGTLVTVSS

SEQ ID NO: 2 - protein, 110 aa, synthetic construct
QSVLTQPASVSGSPGQSITISCAGTSSDVGGSSYVSWYQQHPGKAPKLMIYYDSYRPSGVSNRFSGSKSGNTASLTISGLQAEDEADYYCSSNTYSSTRVFGGGTKLAVL

SEQ ID NO: 3 - protein, 118 aa, synthetic construct
EVQLVESGGSLVKPGGSLRLSCAASGFTFSNNYMNWVRQAPGKGLEWISGIDGSSRGIDYADFVKGRFTISRDNATNSLYLQMNSLRAEDTAVYYCVRSSSNGGMDVWGRGTLVTVSS

SEQ ID NO: 4 - protein, 110 aa, synthetic construct
QSVLTQPASVSGSPGQSITISCAGTSSDVGGNGYVSWYQQHPGKAPKLMIYYDSYRPSGVSNRFSGSKSGNTASLTISGLQAEDEADYYCSSYRSGSTRVFGGGTKLAVL

SEQ ID NO: 5 - protein, 118 aa, synthetic construct
EVQLVESGGSLVKPGGSLRLSCAASGFTFSNYDMNWVRQAPGKGLEWISSISGSSRDIYYADFVKGRFTISRDNATNSLYLQMNSLRAEDTAVYYCVRSSYYYGMDVWGRGTLVTVSS

SEQ ID NO: 6 - protein, 110 aa, synthetic construct
QSVLTQPASVSGSPGQSITISCAGTSSDVGGYYGVSWYQQHPGKAPKLMIYYDSNRPSGVSNRFSGSKSGNTASLTISGLQAEDEADYYCSSYTYNSTRVFGGGTKLAVL

SEQ ID NO: 7 - protein, 120 aa, synthetic construct
EVQLVESGGSLVKPGGSLRLSCAASGFTFSNYSMNWVRQAPGKGLEWISSISGSSRSISYADFVKGRFTISRDNATNSLYLQMNSLRAEDTAVYYCVRSSYYGGNGMDVWGRGTLVTVSS

SEQ ID NO: 8 - protein, 110 aa, synthetic construct
QSVLTQPASVSGSPGQSITISCAGTSSDVGGSYYVSWYQQHPGKAPKLMIYGDSYRPSGVSNRFSGSKSGNTASLTISGLQAEDEADYYCSSYTYYSTRVFGGGTKLAVL